(19) United States
(12) Patent Application Publication
(10) Pub. No.: US 2017/0266123 A1
(43) Pub. Date: Sep. 21, 2017

(54) COATED PARTICLES FOR DRUG DELIVERY

(71) Applicant: Dana-Farber Cancer Institute, Inc., Boston, MA (US)

(72) Inventors: Zohreh Amoozgar, Brighton, MA (US); Michael Solomon Goldberg, Brookline, MA (US)

(73) Assignee: Dana-Farber Cancer Institute, Inc., Boston, MA (US)

(21) Appl. No.: 15/503,766

(22) PCT Filed: Aug. 14, 2015
(86) PCT No.: PCT/US15/45417

§ 371 Date: Feb. 14, 2017

Related U.S. Application Data
(60) Provisional application No. 62/037,556, filed on Aug. 14, 2014.

(51) Int. Cl. A61K 9/51; A61K 31/337; A61K 39/395; A61K 38/46; A61K 38/48; A61K 31/4439; A61K 38/47

(52) U.S. Cl. CPC A61K 9/5146; A61K 9/5153; A61K 9/5192; A61K 31/4439; A61K 31/337; A61K 38/47; C12Y 302/01017; A61K 38/465; A61K 38/4886; A61K 39/3955; A61K 2039/505

(57) ABSTRACT

In one aspect, a particle comprising a core containing at least one pharmaceutically active agent and a coating covering the surface of the particle that comprises a biocompatible adhesive polymer is provided. The core may comprise two or more components, such as two pharmaceutically active agents or a pharmaceutically active agent and a major constituent of the core, having at least one dissimilar chemical or physical property (e.g., molecular weight, solubility, c Log P). In some such embodiments, placement of the uncoated core in certain environments results in the rapid release of a component (e.g., a pharmaceutically active agent) from and/or destabilization and breakdown of the core. In some embodiments, the biocompatible adhesive polymer in the coating acts as a molecular glue to stabilize the core and/or alter the release kinetics of at least one pharmaceutically active agent.

### RELATED APPLICATIONS

The present application claims priority under 35 U.S.C. §119(e) to U.S. provisional patent application, U.S. Ser. No. 62/037,556, entitled “Coated Particle for Drug Delivery,” filed Aug. 14, 2014, which is incorporated herein by reference.

### FIELD OF INVENTION

The present invention relates to coated particles for drug delivery, compositions thereof, methods for preparing such particles, and uses for the treatment of disease.

### BACKGROUND

Particles are often used as delivery systems for pharmaceutically active agents. The use of nanoparticles allows the pharmaceutically active agent to be transported to and/or accumulate at the target site (i.e., the place of action), thereby minimizing undesirable side effects and lowering the required therapeutic dose. Moreover, encapsulation of pharmaceutically active agents in particles greatly enhances the therapeutic window of many pharmaceutically active agents, thereby reducing the frequency of administration. Certain applications require the particles to be biocompatible, stable in physiological conditions, exhibit sustained or controlled release kinetics, and have a relatively small diameter. Conventional methods for forming such particles can be complex, costly, or incompatible with certain classes of pharmaceutically active agents. Accordingly, improved compositions and methods for preparing particles are needed.

### SUMMARY

The present invention provides methods for forming particles as well as the particles themselves, compositions, preparations, formulations, and kits useful for administration to a subject. The subject matter of the present invention involves, in some cases, interrelated products, alternative solutions to a particular problem, and/or a plurality of different uses of one or more systems and/or articles.

In one aspect, a nanoparticle comprising a polymeric core containing at least one pharmaceutically active agent and a coating covering the surface of the particle that comprises a biocompatible adhesive polymer is provided. In some embodiments, the methods described herein, allow two or more components, such as two pharmaceutically active agents or a pharmaceutically active agent and a major constituent of the core (e.g., the polymer), having at least one dissimilar chemical or physical property (e.g., molecular weight, solubility, c Log P) to be formed into a relatively stable, biocompatible particle exhibiting sustained release kinetics of the pharmaceutically active agent(s). Conventional particles comprising components having dissimilar chemical and/or physical properties are often unable to be both biocompatible and exhibit the requisite stability to achieve sustained or controlled release of one or more pharmaceutically active agents under physiological conditions without utilizing complex techniques, expensive processes, and/or harsh chemical treatments. These conventional particles are often unable to encapsulate multiple pharmaceutically active agents in a single particle and thereby limit the development of localized multi-drug therapies delivered in a single dosage form. Moreover, certain pharmaceutically active agents cannot be delivered with the desired release kinetics from these conventional particles, because the limited number of biocompatible materials that would be sufficiently similar to the pharmaceutically active agent to form a stable particle cannot produce the desired release kinetics. In some embodiments, the coated particles, described herein, are resistant to the relatively rapid destabilization and/or drug release that occurs due to a dissimilar chemical or physical property between two or more components.

In another aspect, a nanoparticle comprises a solid core, a coating covering the surface of the polymeric core, and a biological macromolecule directly attached to the surface of the coating, wherein the coating comprises a biocompatible adhesive polymer comprises a catechol-containing repeating unit.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### Definitions

“Antibody”: The term “antibody” refers to an immunoglobulin, whether natural or wholly or partially synthetically produced. All derivatives or fragments thereof which maintain specific binding ability are also included in the term. The term also covers any protein having a binding domain which is homologous or largely homologous to an immunoglobulin binding domain. An antibody may be monoclonal or polyclonal. The antibody may be a member of any immunoglobulin class, including any of the human classes: IgG, IgM, IgA, IgD, and IgE. In certain embodiments, antibodies of the IgG class are used.

“Antibody fragment”: The term “antibody fragment” refers to any derivative of an antibody which is less than full-length. Examples of antibody fragments include, but are not limited to, Fab, Fab′, F(ab′)₂, scFv, Fv, dsFv diabody, Fc, and Fd fragments. In certain embodiments, the fragment is an Fc fragment, more particularly an Fc fragment of an IgG antibody. The antibody fragment may be produced by any means. For instance, the antibody fragment may be enzymatically or chemically produced by fragmentation of an intact antibody, or it may be recombinantly produced from a gene encoding a partial antibody sequence. Alternatively, the antibody fragment may be wholly or partially synthetically produced. The antibody fragment may be a single chain antibody fragment. A functional antibody fragment will typically comprise at least about 50 amino acids and more typically will comprise at least about 200 amino acids.

“Administer”: The terms “administer,” “administering,” or “administration,” as used herein, refers to implanting, absorbing, ingesting, injecting, inhaling, or otherwise introducing an inventive compound, or a composition thereof, in or on a subject.

“Biocompatible: As used herein, the term “biocompatible” is intended to describe a material (e.g., particles, excipients) that is not toxic to cells. Particles are “biocompatible” if their addition to cells in vitro results in less than 20% (e.g., less than 15%, less than 10%, less than 5%, less than 3%, less than 2%, less than 1%) cell death, and their administration in vivo does not induce inflammation or other such adverse effects.

“Biodegradable”: As used herein, “biodegradable” compounds are those that, when introduced into cells, are broken down by the cellular machinery or by hydrolysis into components that the cells can either reuse or dispose of without significant toxic effects on the cells (i.e., fewer than about 20% of the cells are killed when the components are added to cells in vitro). The components preferably do not induce inflammation or other adverse effects in vivo. In certain embodiments, the chemical reactions relied upon to break down the biodegradable compounds are uncatalyzed. For example, the inventive materials may be broken down in part by the hydrolysis of the polymeric material of the inventive coated particles.

“Biological macromolecule” or “biomacromolecule”: The terms biological macromolecule and biomacromolecule refers to a macromolecule comprising at least 10 (e.g., at least 15, at least 25, at least 50) sugar, amino acid, and/or nucleotide repeat units. The biological molecule may be capable of undergoing a biological binding event (e.g., between complementary pairs of biological molecules) with another biological molecule. The biological macromolecule may be a nucleic acid, protein, peptide, or carbohydrate.

“Composition”: The terms “composition” and “formulation” are used interchangeably.

“Condition”: As used herein, the terms “condition,” “disease,” and “disorder” are used interchangeably.

“Particle”: As used herein, the term “particle” refers to a small object, fragment, or piece of material and includes, without limitation, microparticles and nanoparticles. Particles may be composed of a single substance or multiple substances. In certain embodiments, the particles are substantially solid throughout and/or comprise a core that is substantially solid throughout. In some embodiments, a particle may not include a micelle, a liposome, or an emulsion. The term “nanoparticle” refers to a particle having a characteristic dimension (e.g., greatest dimension, average diameter) of less than about 1 micrometer and at least about 1 nanometer, where the characteristic dimension of the particle is the largest cross-sectional dimension of the particle. The term “microparticle” refers to a particle having a characteristic dimension of less than about 1 millimeter and at least about 1 micrometer, where the characteristic dimension of the particle is the smallest cross-sectional dimension of the particle.

“Pharmaceutically active agent”: As used herein, the term “pharmaceutically active agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Pharmaceutically active agents include, without limitation, agents listed in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005. Preferably, though not necessarily, the pharmaceutically active agent is one that has already been deemed safe and effective for use in humans or animals by the appropriate governmental agency or regulatory body. For example, drugs approved for human use are listed by the FDA under 21 C.F.R. §§330.5, 331 through 361, and 440 through 460, incorporated herein by reference; drugs for veterinary use are listed by the FDA under 21 C.F.R. §§500 through 589, incorporated herein by reference. All listed drugs are considered acceptable for use in accordance with the present invention. In certain embodiments, the pharmaceutically active agent is a small molecule. Exemplary pharmaceutically active agents include, but are not limited to, anti-cancer agents, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, prostaglandins, etc.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Small molecule”: As used herein, the term “small molecule” refers to pharmaceutically active agent, whether naturally-occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, acyls, and heterocyclic rings, etc.). In certain embodiments, the molecular weight of a small molecule is at most about 2,500 g/mol, is at most about 2,000 g/mol, at most about 1,500 g/mol, at most about 1,250 g/mol, at most about 1,000 g/mol, at most about 900 g/mol, at most about 800 g/mol, at most about 700 g/mol, at most about 600 g/mol, at most about 500 g/mol, at most about 400 g/mol, at most about 300 g/mol, at most about 200 g/mol, or at most about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and at most about 2,500 g/mol, at least about 200 g/mol and at most about 2,000 g/mol, at least about 200 g/mol and at most about 1,500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions.

“SB-431542”: As used herein, “SB-431542” refers to the drug developed by GlaxoSmithKline that is an inhibitor of the activin receptor-like kinase receptors, such as ALK5, ALK4, and ALK7. SB-431542 has the structure:

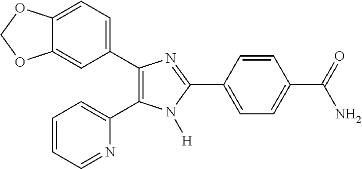 [SMILES: NC(=O)c1ccc(-c2nc(-c3ccc4c(c3)OCO4)c(-c3ccccn3)[nH]2)cc1]

“Solubility:” As used herein, “solubility” refers to the ability of a molecule to be carried in the solvent without precipitating out. The solubility may be expressed in terms of concentration of the saturated solution of the molecule at standard conditions.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. The animal may be a transgenic animal or genetically engineered animal. In certain embodiments, the subject is non-human animal. In certain embodiments, the animal is fish. A “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

“Surface modifying agents”: As used herein, the term “surface modifying agent” refers to any chemical compound that can be attached to the surface of a particle. The surface modifying agent may be any type of chemical compound including small molecules, polynculeotides, proteins, peptides, metals, polymers, oligomers, organometallic complexes, lipids, carbohydrates, etc. The agent may modify any property of particle including surface charge, hydrophilicity, hydrophobicity, zeta potential, size, thickness of coating, etc. In certain embodiments, the surface modifying agent is a polymer such as polyethylene glycol (PEG) or co-polymers thereof.

As defined herein, the term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. A “non-target tissue” is any biological tissue of a subject (including a group of cells, a body part, or an organ) or a part thereof, including blood and/or lymph vessels, which is not a target tissue.

“Targeting moiety”: The term “targeting moiety” refers to a chemical moiety that facilitates localization to a particular targeting site, such as a tumor, a disease site, a tissue, an organ, a type of cell, or an organelle, and is able to bind to or otherwise associate with a biological moiety, for example, a membrane component, a cell surface receptor, organelle component, or the like. The targeting moiety may be directly bound to the particle or may be associated with the particle through a linking moiety.

“Therapeutically effective amount”: As used herein, and unless otherwise specified, a “therapeutically effective amount” of a compound is an amount sufficient to provide a therapeutic benefit in the treatment of a disease, disorder, or condition, or to delay or minimize one or more symptoms associated with the disease, disorder, or condition. A therapeutically effective amount of a compound means an amount of therapeutic agent, alone or in combination with other therapies, which provides a therapeutic benefit in the treatment of the disease, disorder, or condition. The term “therapeutically effective amount” can encompass an amount that improves overall therapy, reduces or avoids symptoms or causes of disease or condition, or enhances the therapeutic efficacy of another therapeutic agent.

“Treatment”: As used herein, the terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1B are (A) a schematic of the release of an pharmaceutically active agent from conventional particles and (B) a schematic of the release of an pharmaceutically active agent from a particle according to certain embodiments of the present invention.

FIGS. 2A-2B are (A) a schematic of the release of pharmaceutically active agents from conventional particles and (B) a schematic of the release of pharmaceutically active agents from a particle according to certain embodiments of the present invention.

FIGS. 3A-3C provide (A) confocal images of J774A. 1 macrophages incubated with polymeric nanoparticles (P NP), polydopamine-coated polymeric nanoparticles (PD NP), or PEGylated polydopamine-coated polymer nanoparticles (PDP NP) for 1 hours, (B) a histogram of the amount of protein adsorbed on polymer particles having a surface modified with PEG, 4arm-PEG-NH₂ (5 kDa), NH₂-PEG-NH₂ (7.5 kDa), 4arm-PEG-NH₂ (10 kDa), Methoxy-PEG-NH₂ (5 kDa), 8arm-PEG-NH₂ (20 kDa), and Y-PEG-NH₂ (40 kDa), and (C) a histogram of cell number for particles having a surface modified with PEG, 4arm-PEG-NH₂ (5 kDa), NH₂-PEG-NH₂ (7.5 kDa), 4arm-PEG-NH₂ (10 kDa), Methoxy-PEG-NH₂ (5 kDa), 8arm-PEG-NH₂ (20 kDa), and Y-PEG-NH₂ (40 kDa).

FIGS. 4A-4B provide (A) scanning electron microscope images of polymeric nanoparticles and (B) PEGylated polydopamine-coated polymeric nanoparticles.

FIGS. 5A-5B are graphs of percent mRNA inhibition of (A) collagen and (B) fibronectin markers versus concentration of SB-431542 and PEGylated polydopamine-coated polymeric nanoparticles containing SB-431542.

FIG. 6 is a graph of percent cell viability for cells exposed to various concentrations of paclitaxel, PEGylated polydopamine-coated polymeric nanoparticles containing paclitaxel, or PEGylated polydopamine-coated polymeric nanoparticles containing paclitaxel and collagenase for 72 hours.

FIG. 7 is a histograph of absorption indicating E-selectin expression after 4 hours in the presence and absence of TNFα.

FIG. 8 is a histograph of fluorescence indicating HL-60 binding of HUVEC cells treated with PEGylated polydopamine-coated polymeric nanoparticles, PEGylated polydopamine-coated polymeric nanoparticles containing an E-selectin antibody, PEGylated polydopamine-coated polymeric nanoparticles containing an E-selectin antibody and SB-431542, or an E-selectin antibody.

FIG. 9 is a graph of cumulative release of paclitaxel versus time for polymeric nanoparticles containing paclitaxel, polydopamine-coated polymeric nanoparticles containing paclitaxel, and PEGylated polydopamine-coated polymeric nanoparticles containing paclitaxel.

FIG. 10 is a graph cell viability for BR5FVB1-Akt cells exposed to paclitaxel and various nanoparticles containing paclitaxel for 72 or 3 hours in a dose-dependent manner.

FIG. 11 is a graph of paclitaxel (PTX) concentration in the blood and peritoneal lavage fluid 3 hours after free PTX or PTX-PDP NP treatment.

FIG. 12 is a graph percent survival of ovarian tumor-bearing mice treated with PTX (, 5 mg/kg dissolved in 50 μL DMSO and 450 μL PBS) and PTX-PDP NP (, 5 mg/kg. p<0.001.

FIG. 13 is a schematic of surface modification of a coated particle with biological macromolecules, according to certain embodiments.

FIGS. 14A-14D are (A) a histogram of particle size and zeta potential for various nanoparticles, (B) a histogram of protein coating percent efficiency and PEG coating percent efficiency for various nanoparticles, (C) a histogram of percentage of stable nanoparticles for various nanoparticles, and (D) a histogram of percentage of functional nanoparticles for various nanoparticles.

FIGS. 15A-15D are (A) a histogram of activity equivalence for free lysozyme, polydopamine coated particles, and lysozyme coated particles, (B) gel electrophoresis images for DNase nanoparticles at a dosage of equivalent to 1 unit DNase and 5 unit DNase and DNase in solution, (C) the results of the adhesion-inhibition assay for anti-CD62E antibody coated nanoparticles, polydopamine nanoparticles, and anti-CD62E antibody in solution, and (D) a graph of activity equivalence for collagenase I coated nanoparticles, polydopamine nanoparticles, and collagenase I in solution.

FIGS. 16A-16E are (A) a histogram of the percent collagen in mice having triple negative breast cancer after intratumoral injection of collagenase I coated nanoparticles, collagenase in solution, and polydopamine coated nanoparticles, (B) a graph of tumor volume versus number of days for collagenase I coated nanoparticles, collagenase in solution, and polydopamine coated nanoparticles, (C) a histogram of the doxorubicin concentration in triple negative breast cancer tumors in mice for collagenase I coated nanoparticles, collagenase in solution, and polydopamine coated nanoparticles, (D) a histogram of the doxorubicin concentration in various tissues of mice having triple negative breast cancer for collagenase I coated nanoparticles, collagenase in solution, and polydopamine coated nanoparticles, and (E) a histogram of the uptake of doxorubicin in M2 macrophages in triple negative breast cancer tumor in mice for collagenase I coated nanoparticles, collagenase in solution, and polydopamine coated nanoparticles.

FIGS. 17A-17C are (A) a schematic of an experimental method for nanoparticles comprising doxorubicin and doxorubicin in solution, (B) a histogram of cell viability for Dox in solution, uncoated nanoparticles comprising doxorubicin (Dox), polydopamine nanoparticles comprising Dox, DNase nanoparticles comprising Dox, and collagenase I nanoparticles comprising Dox at various times, and (C) a histogram of the number of mammospheres for untreated 4T1 cells, Dox in solution, DNase nanoparticles comprising Dox, and collagenase I nanoparticles comprising Dox.

### DETAILED DESCRIPTION

In one aspect, a particle comprising a core containing at least one pharmaceutically active agent and a coating covering the surface of the particle that comprises a biocompatible adhesive polymer is provided. The core may comprise two or more components, such as two pharmaceutically active agents or a pharmaceutically active agent and a major constituent of the core, having at least one dissimilar chemical or physical property (e.g., molecular weight, solubility, c Log P). In some such embodiments, placement of the uncoated core in certain environments results in the rapid release of a component (e.g., a pharmaceutically active agent) from and/or destabilization and breakdown of the core. For example, a hydrophobic polymer forming the uncoated core and a hydrophilic pharmaceutically active agent within the uncoated core may have such different solubilities that placement of the uncoated core in an aqueous environment causes the pharmaceutically active agent to be rapidly released from the uncoated core. In some embodiments, the biocompatible adhesive polymer in the coating acts as a molecular glue to stabilize the core and/or alter the release kinetics of at least one pharmaceutically active agent. Thus, in some embodiments, coated particles, as described herein, are resistant to the relatively rapid destabilization and/or drug release that occurs due to a dissimilar chemical or physical property between, e.g., a major component of the core (e.g., a polymer) and a pharmaceutically active agent or between two or more pharmaceutically active agents.

Many applications require particulate delivery systems to be biocompatible and exhibit sustained or controlled release of the pharmaceutically active agent under physiological conditions. The release characteristics of these systems are dependent on particle stability, amongst other factors. Particles that are uncontrollably destabilized under physiological conditions may release their cargo prematurely, release a quantity of pharmaceutically active agent that is at or above the minimum toxic concentration, and/or have a relatively short release duration. Stability of particles is influenced by the compatibility of the components used to form the particles, since components having dissimilar chemical and/or physical properties may readily dissociate from each other destabilizing the particle and/or resulting in the rapid release of one or more component of the particle. However, due to the diversity of pharmaceutically active agents, it is often difficult to find a biocompatible material with sufficient similarity to the pharmaceutically active agent to form particles having the requisite stability and that would otherwise exhibit the desired release kinetics. Furthermore, the selection of appropriate biocompatible materials to contain two or more pharmaceutically active agents having different chemical and/or physical properties is especially difficult.

Therefore, some conventional particles comprising components having dissimilar chemical and/or physical properties are unable to be both biocompatible and exhibit the requisite stability to achieve sustained or controlled release of one or more pharmaceutically active agents under physiological conditions. These conventional particles are unable to encapsulate multiple pharmaceutically active agents in a single particle and thereby development of localized multi-drug therapies that are delivered in a single dosage form (e.g., single nanoparticle). Moreover, certain pharmaceutically active agents may not be able to be delivered with the desired release kinetics from these conventional particles due to the inability to select a biocompatible material that would be sufficiently similar to the pharmaceutically active agent to form a stable particle and that would otherwise produce the desired release kinetics.

Other conventional methods for producing biocompatible particles have tried to address this problem by utilizing complex techniques, expensive processes, harsh chemical treatments, and/or methods that are incompatible with certain classes of pharmaceutically active agents to control the stability and release characteristics of particles comprising two or more components having a dissimilar chemical or physical property. It has been discovered, within the context of certain embodiments of the present invention, that coating a particle with a biocompatible adhesive polymer improves the stability and/or alters the release kinetics of particles, including those having two or more dissimilar components.

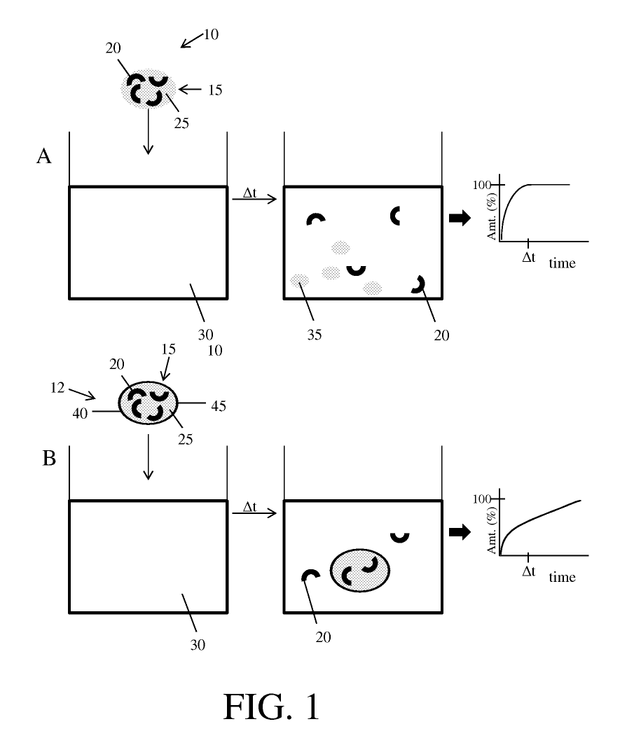

A non-limiting example of a conventional particle and a particle, as described herein, is shown in FIG. 1A and FIG. 1B, respectively. As schematically illustrated in FIG. 1A, a conventional particle 10 may comprise a core 15 containing a pharmaceutically active agent 20 (e.g., biological macromolecule). At least a portion of the core may be formed from a component 25 (e.g., polymer). The component 25 and the pharmaceutically active agent 20 may have at least one dissimilar chemical or physical property. For example, the component 25 may be hydrophobic and/or have a low solubility in the environment 30. The pharmaceutically active agent 25 may be hydrophilic and/or have a relatively high solubility in the environment. When the particle 10 is placed in an environment 30 (e.g., aqueous-based environment), over a relatively short period of time (Δt) the pharmaceutically active agent may separate from the component resulting in the rapid release of the pharmaceutically active agent and breakdown of the core into pieces 35.

Conversely, as illustrated in FIG. 1B, the particle 12 having the same core 15 as particle 10 containing a pharmaceutically active agent 20 may be coated with a coating 40 covering at least a portion of the surface 45 of the particle 10. The coating may comprise a biocompatible adhesive polymer, such as a polymer comprising a catechol-containing repeat unit. When the coated particle is placed in the same environment 30, the particle may be stable over the time Δt and/or have different release characteristics than the conventional particle. For example, as shown in FIG. 1A, the conventional particle may exhibit burst release kinetics resulting in a relatively large amount of the pharmaceutically active agent being delivered over a relatively short duration, such that at least about 50% (e.g., at least about 60%, at least about 70%, at least about 80%, at least about 90%, at least about 95%) or 100%, as illustrated, of the pharmaceutically active agent is released by time Δt. In some instances, the amount delivered may result in a concentration that is at or above the minimum toxic concentration and cause undesired effects. In other instances, the short release duration may necessitate relatively frequent administration to maintain the pharmaceutically active agent within the desired (e.g., therapeutic) range. However, as shown in FIG. 1B, the coated particle may exhibit sustained release of the pharmaceutically active agent 25, such that amount of the pharmaceutically active agent released at time Δt is significantly lower than the uncoated particle. For instance, in some embodiments, the coating increases the duration of release by at least about 1.25 times (e.g., at least about 1.5 times, at least about 2 times, at least about 4 times, at least about 5 times, at least about 10 times, at least about 15 times, at least about 25 times, at least about 50 times, at least about 100 times). In some instances, the increase in duration ranges from at least about 1.25 times to at most about 200 times (e.g., at least about 2 times to at most about 200 times, at least about 5 times to at most about 200 times, at least about 10 times to at most about 200 times, at least about 25 times to at most about 200 times).

It should be understood that Δt may vary based on the components (e.g., polymer, pharmaceutically active agents) in the particle. In some instances Δt may be on the order of seconds, minutes, or hours.

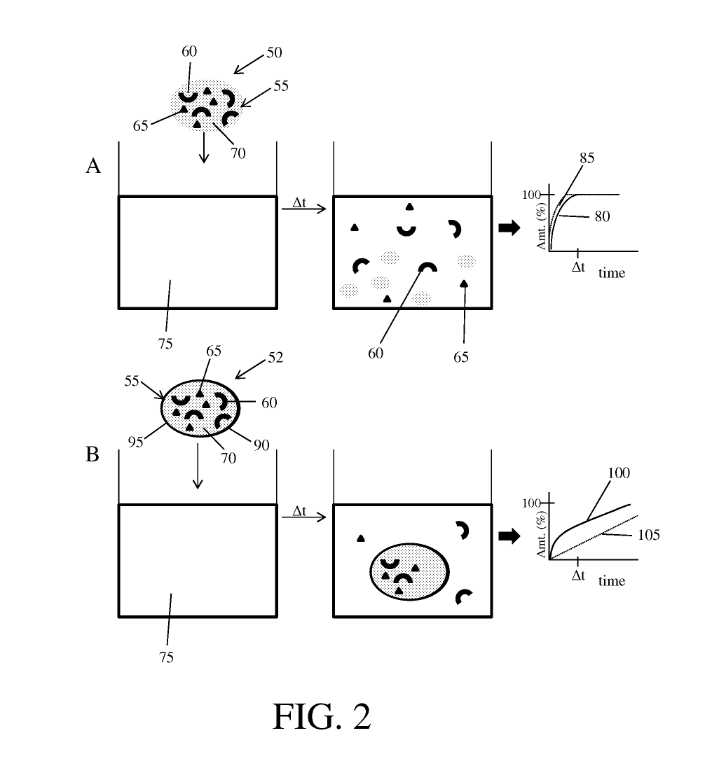

Another non-limiting example of a conventional particle and a particle, as described herein, is shown in FIG. 2A and FIG. 2B, respectively. In some embodiments, as schematically illustrated in FIG. 2A, a conventional particle 50 may comprise a first pharmaceutically active agent (e.g., a biological macromolecule) 60 and a second pharmaceutically active agent (e.g., a small molecule) 65. At least a portion of the core 55 may be formed from a component 70 (e.g., a polymer). Two or more components (e.g., 60, 65, 70) of the core may have at least one dissimilar chemical or physical property. For instance, the component 70 and the first pharmaceutically active agent may have a dissimilar chemical or physical property that negatively influences the stability of the core and/or release characteristics under certain conditions. In another example, the component 70 and the second pharmaceutically active agent may have a dissimilar chemical or physical property. In some embodiments, as illustrated in FIG. 2A, the first pharmaceutically active agent and the second pharmaceutically active agent may have at least one dissimilar chemical or physical property. For instances, the first and second pharmaceutically active agents may have different solubilities in the desired environments and/or very different molecular weights. In some instances, biocompatible core forming components compatible with both the first and second pharmaceutically active agents may not be readily available or suitable for the intended use of the particle. In some such embodiments, one or more biocompatible components used to form the core may be selected to be compatible with the first pharmaceutically active agent, the second pharmaceutically active agent, or neither agent.

As illustrated in FIG. 2A, when the particle 50 is placed in an environment 75 (e.g., aqueous-based environment), over a relatively short period of time (Δt) at least one component in the core may dissociate from another component in the core resulting in particle destabilization and/or rapid release of one or more component from the particle. For example, the first pharmaceutically active agent may separate from the second pharmaceutically active agent, the first pharmaceutically active agent may separate from component 70, and/or the second pharmaceutically active agent may separate from component 70. In some cases, the conventional particle may exhibit burst release kinetics of both pharmaceutically active agents resulting in a relatively large amount of the pharmaceutically active agents being delivered over a relatively short release duration; in some instances, at least about 50% (e.g., at least about 60%, at least about 70%, at least about 80%, at least about 90%, at least about 95%) or 100%, as illustrated in FIG. 2A, of the pharmaceutically active agents are released by time Δt. In some embodiments, particle destabilization or component dissociation may dominate the release characteristics of the pharmaceutically active agents, such that the release profile 80 of the first pharmaceutically active agent may be substantially the same as the release profile 85 of the second pharmaceutically active agent. In other embodiments, the release profiles of the first and second pharmaceutically active agents may be different, though at least one pharmaceutically active agent may still have a relatively short release duration.

Conversely, as illustrated in FIG. 2B, the particle 52 comprising the same core 55 as particle 50 containing a first pharmaceutically active agent (e.g., biological macromolecule) 60 and a second pharmaceutically active agent (e.g., small molecule) 65 may be coated with a coating 90 covering at least a portion of the surface 95 of the particle 50. When the coated particle is placed in the same environment 75, the particle may be stable over the time Δt and/or have different release characteristics than the conventional particle. As shown in FIG. 2B, the coated particle may exhibit sustained release of both pharmaceutically active agents, such that amount of the first and/or second pharmaceutically active agent released at time Δt is significantly lower than the uncoated particle. For instance, in some embodiments, the coating increases the duration of release of the first and/or second pharmaceutically active agent by at least about 1.25 times (e.g., at least about 1.5 times, at least about 2 times, at least about 4 times, at least about 5 times, at least about 10 times, at least about 15 times, at least about 25 times, at least about 50 times, at least about 100 times). In some instances, the increase in duration ranges from at least about 1.25 to at most about 200 times (e.g., at least about 2 times to at most about 200 times, at least about 5 times to at most about 200 times, at least about 10 times to at most about 200 times, at least about 25 times to at most about 200 times). Moreover, as illustrated in FIG. 2B, since the release characteristics of the pharmaceutically active agent is not dominated by particle destabilization and/or component dissociation, the first and second pharmaceutically active agents may have different release profiles 100 and 105, respectively. In other instances, the first and second pharmaceutically active agents may have substantially similar release characteristics.

As described herein, a particle may comprise a core containing two or more components having at least one dissimilar chemical or physical property. As used herein, a dissimilar chemical or physical property refers to a chemical or physical property of the component that is sufficiently different from another component that the dissimilar components readily dissociate under certain conditions and/or at least one of the dissimilar components is readily released from the core. This ready dissociation or release of a component may affect the stability of the core. Non-limiting examples of chemical and physical properties that may influence the stability of the core or its release characteristics include hydrophobicity, molecular weight, pKa, c Log P, surface change, ability to form intramolecular non-covalent interactions (e.g., hydrogen bonding), and solubility in a particular solvents. Those of ordinary skill in the art would be aware of other chemical or physical properties that may influence the stability of the core or its release characteristics.

For instance, in some embodiments, the dissimilar chemical property may be solubility in a particular solvent. In certain embodiments, the solubility of a core component (e.g., first pharmaceutically active agent), in at least one solvent, is substantially different than the solubility of another core component (e.g., second pharmaceutically active agent or polymer). For example, an pharmaceutically active agent may have a relatively low solubility (e.g., solubility of less than about 1 mg/ml) in a solvent (e.g., aqueous based solvent), and a core-forming polymer and/or second pharmaceutically active agent may have a relatively high solubility (e.g., greater than about 10 mg/ml) in the solvent (e.g. aqueous based solvent) at standard temperature and pressure. In some such embodiments, the pharmaceutically active agent may readily dissociate from the other core component when placed in the solvent or an environment comprising the solvent.

In some such embodiments, the solubility of the pharmaceutically active agent may be at least about 2 times, at least about 5 times, at least about 10 times, at least about 25 times, at least about 50 times, at least about 75 times, at least about 100 times, at least about 150 times, at least about 200 times, at least about 250 times, at least about 300 times, at least about 350 times, at least about 400 times, at least about 450 times, or at least about 500 times greater than the solubility of another core component (e.g., second pharmaceutically active agent, core-forming polymer). In some instances, the solubility of the pharmaceutically active agent ranges from at least about 2 to at most about 1,000 times (e.g., at least about 5 times to at most about 1,000 times, at least about 10 times to at most about 1,000 times, at least about 50 times to at most about 1,000 times, at least about 100 times to at most about 1,000 times) greater than the solubility of another core component. For example, when the solvent is aqueous based, the solubility of a hydrophilic pharmaceutically active agent (e.g., biological macromolecule) is at least 10 times (e.g., at least 25 times, at least 50 times, at least 100 times, or at least 200 times) greater than a hydrophobic pharmaceutically active agent (e.g., small molecule drug) or hydrophobic polymer.

For instance, in some embodiments, the solubility of the pharmaceutically active agent in the solvent (e.g., water, ethanol) may be greater than or equal to about 0.01 g/ml, greater than or equal to about 0.05 g/ml, greater than or equal to about 0.1 g/ml, greater than or equal to about 0.5 g/ml, greater than or equal to about 1.0 g/ml, greater than or equal to about 5 g/ml, greater than or equal to about 10 g/ml, greater than or equal to about 25 g/ml, greater than or equal to about 50 g/ml, or greater than or equal to about 75 g/ml. In some instances, the solubility of another core component (e.g., second pharmaceutically active agent, core-forming polymer) in the same solvent is less than or equal to about 1 mg/ml, less than or equal to about 0.75 mg/ml, less than or equal to about 0.5 mg/ml, less than or equal to about 0.25 mg/ml, less than or equal to about 0.1 mg/ml, less than or equal to about 0.05 mg/ml, less than or equal to about 0.01 mg/ml, less than or equal to about 0.005 mg/ml, or less than or equal to about 0.001 mg/ml.

In some embodiments, a dissimilar physical property may be molecular weight. For example, the core may comprise a first pharmaceutically active agent and a second pharmaceutically active agent that have significantly different molecular weights. Many conventional techniques for particle formation are unable to form stable particles exhibiting sustained release of pharmaceutically active agents having significantly different molecular weights without relying on complex techniques, expensive processes, harsh chemical treatments, and/or methods that are incompatible with certain classes of pharmaceutically active agents. However, the methods, described herein, may be used to form relatively stable particles having a core comprises pharmaceutically active agents with significantly different molecular weights. For instance, in some embodiments, the difference in molecular weight between two pharmaceutically active agent contained in the core is at least about 1,000 g/mol, at least about 2,500 g/mol, at least about 5,000 g/mol, at least about 7,500 g/mol, at least about 10,000 g/mol, at least about 25,000 g/mol, at least about 50,000 g/mol, at least about 7,500 g/mol, or at least about 100,000 g/mol. In some instances, the difference in molecular weight is at most about 750,000 g/mol.

In some such embodiments, a first pharmaceutically active agent (e.g., biological macromolecule) has a molecular weight that is at least about 10 times, at least 25 times, at least 50 times, at least 75 times, at least 100 times, at least 150 times, at least 200 times, at least 250 times, at least 300 times, at least 350 times, at least 400 times, at least 450 times, or at least 500 times greater than the molecular weight of a second pharmaceutically active agent (e.g., small molecule) or another core component (e.g., core-forming polymer). In some instances, the first pharmaceutically active agent has a molecular weight that ranges from at least about 2 times to at most about 1,000 times, at least about 5 times to at most about 1,000 times, at least about 10 times to at most about 1,000 times, at least about 50 times to at most about 1,000 times, or at least about 100 times to at most about 1,000 times greater than the molecular weight of a second pharmaceutically active agent. For example, an extracellular matrix degrading enzyme (e.g., metalloproteinase) may have a molecular weight that is at least 10 times (e.g. at least 25 times, at least 50 times, at least 100 times, at least 200 times) greater than a small molecule anticancer agent.

It should be understood that, in certain embodiments, particles need not contain one or more components having dissimilar chemical or physical properties. In some such embodiments, the coating serves to enhance the stability of the particle and/or its release characteristics.

In some embodiments, the core comprises one or more polymers. The core may contain a relatively high weight percentage of polymer(s) and may be referred to as a polymeric core. For instance, in some embodiments, the weight percentage of polymers in the core may be greater than or equal to about 30 wt. %, greater than or equal to about 40 wt. %, greater than or equal to about 50 wt. %, greater than or equal to about 60 wt. %, greater than or equal to about 70 wt. %, greater than or equal to about 80 wt. %, or greater than or equal to about 90 wt. %. In certain embodiments, the polymeric core may consist essentially of one or more polymers and one or more pharmaceutically active agents. In general, any suitable biocompatible polymer may be used to form the core. In some embodiments, the polymers may be selected based on compatibility with one or more pharmaceutically active agent, desired release characteristics, and/or the intended use of the pharmaceutically active agents. For instance, in some embodiments, a polymer may be selected based on its compatibility with pharmaceutical applications and other consumer products (e.g., cosmetics, food).

As described herein, a particle may comprise a coating covering at least a portion of the surface of the core. In some embodiments, the coating may comprise a biocompatible adhesive polymer. The biocompatible adhesive polymer may serve to inhibit particle destabilization and improve release characteristics. Without being bound by theory, it is believed that unlike other polymers, adhesive polymers form such a strong bond with and seal around the particle that destabilization of the particle and/or rapid release of a component of the core becomes energetically unfavorable. Non-limiting examples of biocompatible adhesive polymers include polymers comprising catechol-containing repeat units, biocompatible epoxy polymers, biocompatible cyanoacrylate polymers, and fibrin glue.

In some embodiments, the biocompatible adhesive polymer may be a polymer comprising a catechol-containing repeat unit. The catechol in the catechol-containing repeat unit may be in the polymer backbone and/or a pendant group. In some instances, the catechol-containing repeat unit is in one or more side chains of the polymer. In some embodiments, the catechol-containing repeat unit may be formed from a catechol-containing molecule comprising the structure:

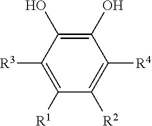

wherein R¹, R², R³ and R⁴ are independently hydrogen, optionally substituted alkyl, optionally substituted heteroalkyl, optionally substituted alkenyl, amine, acyl, and optionally any two R may be joined to form a ring. In some embodiments, the catechol-containing molecule may be a catecholamine-containing molecule. For instance, in some embodiments, at least one of R¹ and R² comprises an amine. In some such embodiments, at least one of R¹ and R² is an optionally substituted heteroalkyl (e.g., optionally substituted alkylamine). In certain embodiments, the catechol-containing molecule is a catecholamine, such as dopamine or an analog thereof.

In some embodiments, the catecholamine comprises the structure:

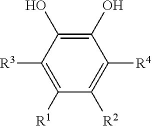

wherein:

R³ is an optionally substituted ethylamine; and

R¹, R², and R⁴ are independently hydrogen, optionally substituted alkyl, optionally substituted alkenyl, amine, acyl, or optionally substituted heteroalkyl; and

optionally any two R may be joined to form a ring.

In some embodiments, the catecholamine may have the structure:

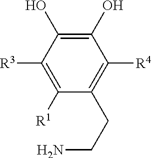

Non-limiting examples of catechol-containing molecules that may be used to form a repeat unit, as described herein, include dopamine and analogs thereof, L-3,4-dihydroxyphenylalanine and analogs thereof, and dihydrocaffeic acid. For example L-3,4-dihydroxyphenylalanine having the structure:

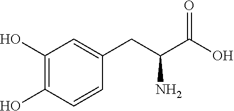

or dihydrocaffeic acid having the structure:

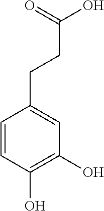 [SMILES: O=C(O)CCc1ccc(O)c(O)c1]

may be used to form a repeat unit.

In some embodiments, the catechol-containing repeat unit comprises the structure:

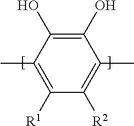

wherein R¹ and R² are independently hydrogen, optionally substituted alkyl, optionally substituted alkenyl, amine, acyl, or optionally substituted heteroalkyl; and optionally R¹ and R² may be joined to form a ring. In some embodiments, the catechol-containing repeat unit may be a catecholamine-containing repeat unit. For instance, in some embodiments, at least one of R¹ and R² comprises an amine. In some such embodiments, at least one of R¹ and R² is an optionally substituted heteroalkyl (e.g., optionally substituted alkylamine).

In some embodiments, the catecholamine repeating unit comprises the structure:

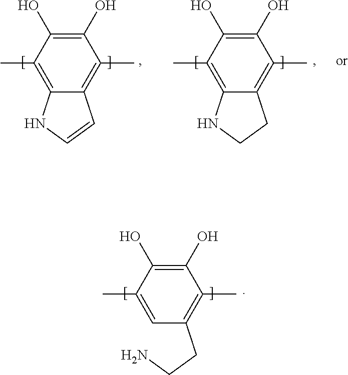

In certain embodiments, the biocompatible adhesive polymer may be polydopamine. Various structures have been reported for polydopamine as described in Hong et al., Adv. Funct. Mater., 2012, 22, 4711-4717, which is incorporated by reference in its entirety. For instance, in some embodiments, the polydopamine may comprise the structure:

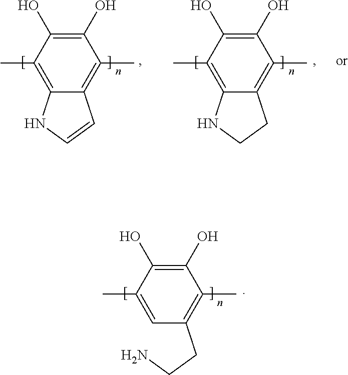

wherein n is 10-1,000.

It should be understood that the catechol containing molecules and polymers described herein may also be provided as homologs, analogs, derivatives, enantiomers, diastereomers, and tautomers of compounds described herein. It will be understood that the skilled artisan will be able to manipulate the conditions in a manner to prepare such homologs, analogs, derivatives, enantiomers, diastereomers, tautomers, and functionally equivalent compounds.

In general, the biocompatible adhesive polymer may have any suitable number of repeat units. For instance, in some embodiments, the number of repeat units in the biocompatible adhesive polymer and/or the number of catechol-containing repeat units may be greater than or equal to about 10, greater than or equal to about 15, greater than or equal to about 20, greater than or equal to about 25, greater than or equal to about 35, greater than or equal to about 50, greater than or equal to about 75, or greater than or equal to about 100. In some instances, the number of repeat units may range from about 10 to about 500, from about 15 to about 500, from about 20 to about 500, from about 25 to about 500, from about 35 to about 500, from about 50 to about 500, from about 75 to about 500, or from about 100 to about 500. The number of repeat units may be determined using gel permeation chromatography (GPC) or nuclear magnetic resonance (NMR).

In some embodiments, the coating may cover a relatively large fraction of the surface of the core. For instance, in some embodiments, greater than or equal to about 40%, greater than or equal to about 50%, greater than or equal to about 70%, greater than or equal to about 80%, greater than or equal to about 90%, greater than or equal to about 95%, greater than or equal to about 97%, or greater than or equal to about 99% of the surface of the particle may be covered with the biocompatible adhesive polymer. The percent coverage of the particle may be determined using scanning electron microscopy techniques.

In some embodiments, the coating may be associated with the core via a chemical and/or biological interaction. In some embodiments, the biocompatible adhesive polymer may associate with the core via a covalent bond or non-covalent interaction (e.g., hydrogen bonding). In some cases, the non-covalent interaction is a hydrogen bond, ionic interaction, dative bond, and/or a Van der Waals interaction. In some embodiments, the core and biocompatible adhesive polymer comprise functional groups capable of forming such bonds. For example, one or more components of the core (e.g., polymer) may include at least one hydrogen atom capable of interacting with a pair of electrons on a hydrogen-bond acceptor in the biocompatible adhesive polymer to form the hydrogen bond. In some embodiments, one or more components of the core (e.g., polymer) may include an electron-rich or electron-poor moiety, such that it may form an electrostatic interaction with the biocompatible adhesive polymer. In some embodiments, an association between the biocompatible adhesive polymer and one or more of the core may occur via a biological binding event (i.e., between complementary pairs of biological molecules). In some embodiments, the association between the core and the coating may depend on the method used to apply the coating.

In some embodiments, the core may be coated with a polymer comprising catechol-containing repeat units (e.g., polydopamine) by immersing the core in a solution (e.g., aqueous solution) containing the catechol-containing monomers (e.g., dopamine) of the polymer. In certain embodiments, the solution may be at an alkaline pH, such as between about 7.5 and about 10, between about 8 and about 10, between about 8 and about 9.5, or between about 8 and about 9. In some embodiments, the catechol-containing monomer may undergo a spontaneous pH-induced oxidative polymerization on the surface of the core to form the polymer comprising catechol-containing repeat units. In some instances, the spontaneous polymerization results in a cross-linked polymer. In certain embodiments, the thickness of the coating may be depend on the amount of time the core is immersed in the solution.

As noted above, the core may contain one or more pharmaceutically active agents. In some embodiments, the core contains a single pharmaceutically active agent (e.g., biological macromolecule, small molecule). For instance, a core formed from a hydrophobic polymer may contain a biological molecule, such as an enzyme or antibody. In some such embodiments, the coating may prevent the relatively rapid release of the biological molecule from the core in aqueous environments. In another example, a core formed from a hydrophobic polymer may contain a relatively hydrophilic small molecule. In such embodiments, the coating may prevent the relatively rapid release of the small molecule from the core in aqueous environments. Alternatively, in another embodiment, a core formed from a hydrophilic polymer may contain a relatively hydrophobic small molecule drug. In such cases, the coating may prevent the relatively rapid dissociation of the small molecule and the polymer forming the core.

In some embodiments, the core contains two or more pharmaceutically active agents. In certain embodiments, the core contains two or more pharmaceutically active agents, such as a small molecule and a biological macromolecule, two or more small molecules, or two or more biological molecules, having similar chemical and physical properties. In certain embodiments, the two or more pharmaceutically active agents may have a different chemical or physical property than a material, which is a major constituent of the core. In some embodiments, the core may contain two or more hydrophobic small molecules drugs. In some such embodiments, the coating may prevent the rapid release or destabilization of the core.

In certain embodiments, the core contains two or more pharmaceutically active agents, such as a small molecule and a biological macromolecule, two or more small molecules, or two or more biological molecules, that are dissimilar with respect to at least one chemical or physical property (e.g., solubility, molecular weight). For example, the core contains a biological molecule and a small molecule that have significantly different molecular weights. For instance, the difference in molecular weight may be at least about 1,000 g/mol, at least about 5,000 g/mol, at least about 10,000 g/mol, or at least about 50,000 g/mol. In another example, the core contains two or more pharmaceutically active agents that have significantly different c Log P or solubility. In some such embodiments, the c Log P may differ by at least about 0.2, at least about 0.5, at least about 0.8, at least about 1.0, at least about 1.25, at least about 1.5, at least about 1.75, at least about 2.0, or at least about 2.25. In yet another example, the core contains two or more pharmaceutically active agents, such as two biological macromolecules, with significantly different pKas and/or surface charge. In some such embodiments, the pKa may differ by at least about 1, differ by at least about 2, differ by at least about 3, differ by at least about 4, differ by at least about 5, or differ by at least about 6 and/or the surface charge may differ by at least about 5 mV, at least about 10 mV, at least about 15 mV, or at least about 20 mV. In some instances in which the pKa and/or surface charge of two pharmaceutically active agents, the agents are oppositely charged at the pH of the delivery environment. In other embodiments, the two pharmaceutically active agents have the same charge.

In some embodiments, the core contains a biological macromolecule (e.g., enzyme, antibody) and a small molecule (e.g., anti-cancer agent). In some embodiments, the core contains a protein and a small molecule. For example, the core contains a protein (e.g., Herceptin, Rituximan, metalloprotease) and an anti-cancer drug (e.g., paclitaxel, doxorubicin). In some instances, the protein is a biotherapeutic. In another example, the core contains an antibody (e.g., E-selectin) and an anti-cancer agent. In embodiments in which the core comprises two or more pharmaceutically active agents, delivery of both pharmaceutically active agents may have a synergistic or additive effect. Moreover, encapsulation of multiple therapeutics in a single particle allows for the delivery of multiple therapies through a single dosage form and thereby potentially broadens the use of multi-drug therapies. In general, the core may contain any suitable number (e.g., one, two, three, four, or more) of pharmaceutically active agents.

In some embodiments, the particles, described herein, may have a relatively small diameter. In certain embodiments, the particle is a nanoparticle. For instance, in some embodiments, the characteristic dimension (e.g., average diameter) of the particles is less than about 1,000 nm, less than or equal to about 800 nm, less than or equal to about 600 nm, less than or equal to about 500 nm, less than or equal to about 400 nm, less than or equal to about 300 nm, less than or equal to about 200 nm, less than or equal to about 100 nm, or less than or equal to about 50 nm. In some instances, the characteristic dimension (e.g., average diameter) of the particles is may be between about 10 nm and about 800 nm, between about 10 nm and about 600 nm, between about 10 nm and about 500 nm, between about 10 nm and about 400 nm, between about 10 nm and about 300 nm, between about 10 nm and about 200 nm, or between about 10 nm and about 100 nm. In some instances, the particles have a diameter less than or equal to 100 nm. In certain cases, the characteristic dimension of the particles is between about 10 nm and about 100 nm. As used herein, the diameter of a particle for a non-spherical particle is the diameter of a perfect mathematical sphere having the same volume as the non-spherical particle. In general, the particles are approximately spherical; however the particles are not necessarily spherical but may assume other shapes (e.g., discs, rods) as well. The measurements described herein typically represent the average particle size of a population. However, in certain embodiments, the measurements may represent the range of sizes found in a population, or the maximum or minimum size of particles found in the population.

In other embodiments, the particle is a microparticle. In certain embodiments, the particles may have an average diameter of less than 1 mm. For instance, in some embodiments, the average diameter of the particles is less than about 1,000 microns, less than or equal to about 500 microns, less than or equal to about 100 microns, less than or equal to about 50 microns, less than or equal to about 10 microns, or less than or equal to about 5 microns and greater than or equal to about 1 micron.

In some embodiments, the diameter of the core may fall within the above-mentioned ranges for the size of the particle. In some such embodiments, the coating may be relatively thin. The thickness of the coating may be controlled by the coating conditions or the coating material used to yield a coated particle with the desired characteristics (e.g., size, zeta potential, biodegradability, stability, etc.). In some embodiments, the thickness of the coating ranges from about 10 nm to about 200 nm, ranging from about 10 nm to about 150 nm, ranges from about 10 nm to about 100 nm, ranges from about 10 nm to about 75 nm, or ranges from about 10 nm to about 50 nm.

In some cases, the particles may have a narrow distribution in a characteristic dimension. For instance, in certain embodiments, the coefficient of variation of a characteristic dimension (e.g., diameter) of the particles may be less than or equal to about 20%, less than or equal to about 15%, less than or equal to about 10%, or less than or equal to about 5%.

Exemplary biological macromolecules include, but are not limited to, proteins (e.g., enzymes, antibodies, glycoproteins), nucleic acids, and polysaccharide (e.g., heparin sulfate), and derivatives or fragments thereof. In some embodiments, the biological molecule is a protein. In some such embodiments, the biological molecule may be an enzyme. The enzyme may be selected from the group consisting of proteases (e.g., serine proteases, threonine proteases, cysteins proteases, aspartate proteases, glutamic acid proteases, metalloproteases), glycosidases (e.g., glucosidase, lactase, amylase, chitinase, sucrase, maltase, neuraminidase, invertase, hyaluronidase and lysozyme), DNAse, and sulfatases. In some instances, the enzyme is an extracellular matrix degrading enzyme, such as a metalloprotease. In some instances, the enzyme is a matrix metalloprotease (e.g., collagenase, gelatinase). As used herein, an extracellular matrix degrading enzyme refers to an enzyme capable of breaking down the extracellular matrix by cleavage of chemical bonds of components of the extracellular matrix such as laminin, fibronectin, proteoglycans, elastin, collagen, glycosaminoglycans (e.g., chondroitin sulfate, heparan sulfate, keratan sulfate, hyaluronic acid). In some embodiments, the biological macromolecule is an antibody or a fragment thereof. In certain embodiments, the biological macromolecule is a cytokine, such as an interleukin. In such cases, the particles containing the cytokine may be used for immunotherapy.

In some embodiments, the weight percentage of a single pharmaceutically active agent (e.g., pharmaceutically active agent) and/or of all the pharmaceutically active agents in the particles (i.e., loading efficiency) is at least about 0.5%, at least about 1%, at least about 2%, at least about 4%, at least about 6%, at least about 8%, at least about 10%, at least about 15%, at least about 20%, at least about 25%, at least about 30%, at least about 35%, at least about 40%, at least about 45%, or at least about 50%. In some instances, the loading efficiency is between about 0.5% and about 60%, between about 0.5% and about 50%, between about 0.5% and about 40%, between about 0.5% and about 30%, between about 1% and about 60%, between about 1% and about 50%, between about 1% and about 40%, between about 1% and about 30%, between about 2% and about 60%, between about 2% and about 50%, between about 2% and about 40%, or between about 2% and about 30%. The loading efficiency may be determined by extracting the pharmaceutically active agent from the dried particles using, e.g., organic solvents, and measuring the quantity of the agent using high pressure liquid chromatography (i.e., HPLC), liquid chromatography-mass spectrometry, nuclear magnetic resonance, or mass spectrometry. Those of ordinary skill in the art would be knowledgeable of techniques to determine the quantity of an agent using the above-referenced techniques. For example, HPLC may be used to quantify the amount of an agent by, e.g., comparing the area under the curve of a HPLC chromatogram to a standard curve.

An exemplary, non-limiting list of polymers that may be used to form the core includes polyesters such as poly(lactic acid)/polylactide, poly(glycolic acid), poly(lactic-co-glycolic acid), and poly(caprolactone); poly(orthoesters); poly(anhydrides); poly(ether esters) such as polydioxanone; poly(carbonates); poly(amino carbonates); and poly(hydroxyalkanoates) such as poly(3-hydroxybutyrate) and poly(3-hydroxybutyrate-co-3-hydroxyvalerate); polyphosphazenes; polyacrylates; poly(alkyl acrylates); polyamides; polyamines such as poly(amido amine) dendrimers; polyethers; poly(ether ketones); poly(alkaline oxides) such as polyethylene glycol; polyacetylenes and polydiacetylenes; polysiloxanes; polyolefins; polystyrene such as sulfonated polystyrene; polycarbamates; polyureas; polyimides; polysulfones; polyurethanes; polyisocyanates; polyacrylonitriles; polysaccharides such as alginate and chitosan; polypeptides; and derivatives and block, random, radial, linear, and teleblock copolymers, and blends of the above. The polymers may be homopolymers or copolymers. Other potentially suitable polymer molecules are described in the Polymer Handbook, Fourth Ed., Brandrup, J. Immergut, E. H., Grulke, E. A., Eds., Wiley-Interscience: 2003, which is incorporated herein by reference in its entirety. In some embodiments, the polymer may be a copolymer.

In some embodiments, the core comprises a synthetic polymer, such as a polyester. For example, the polymer may be poly(lactic-co-glycolic acid). In certain embodiments, the polymer is a block copolymer, such as poly(lactic-co-glycolic acid)-polyethylene glycol block copolymer. In certain embodiments, the core comprises a natural polymer (e.g., polypeptides, polysaccharides, and polynucleotides) or a chemically modified natural polymer. In some embodiments, one or more polymers in the core is biodegradable. In some instances, the core comprises one or more hydrolytically degradable polymers. In other embodiments, the core comprises one or more non-degradable polymers. In embodiments where the polymer matrices are in a composition for administration to a subject, the core typically includes non-toxic and/or bioabsorbable polymers. In some embodiments, the core comprises a mixture of two or more polymers.

The polymers are generally extended molecular structures comprising backbones which optionally contain pendant side groups or chains, wherein the term backbone is given its ordinary meaning as used in the art, e.g., a linear chain of atoms within the polymer by which other chains may be regarded as being pendant. Typically, but not always, the backbone is the longest chain of atoms within the polymer. A polymer may be a co-polymer, for example, a block, alternating, or random co-polymer. Polymers may be obtained from natural sources or be created synthetically. In some embodiments, the polymer may be acyclic or cyclic. A polymer may be cross-linked, for example, through covalent bonds, ionic bonds, hydrophobic bonds, and/or metal binding. In some embodiments, the polymers in the core may not be cross-linked. In other embodiments, at least a portion of the particles in the core may be cross-linked. In embodiments in which at least a portion of the polymers in the core are cross-linked, the polymers in the core may be non-covalently cross-linked. In other embodiments, at least a portion of the particles in the core are covalently cross-linked.

The core may comprise polymers having any suitable molecular weight. For example, in some embodiments, the number average molecular weight of one or more polymers in the core may be greater than or equal to about 3,000 g/mol, greater than or equal to about 5,000 g/mol, greater than or equal to about 10,000 g/mol, greater than or equal to about 25,000 g/mol, greater than or equal to about 50,000 g/mol, about 70,000 g/mol, greater than or equal to about 100,000 g/mol, greater than or equal to about 250,000 g/mol, or greater than or equal to about 500,000 g/mol. In some instances, the number average molecular weight of one or more polymers in the core may be less than or equal to about 1,000,000 g/mol, less than or equal to about 750,000 g/mol, less than or equal to about 500,000 g/mol, less than or equal to about 250,000 g/mol, less than or equal to about 100,000 g/mol, less than or equal to about 75,000 g/mol, less than or equal to about 50,000 g/mol, or less than or equal to about 25,000 g/mol. Combinations of the above-referenced ranges are also possible (e.g., greater than or equal to about 3,000 g/mol and less than or equal to about 1,000,000 g/mol). The number average molecular weight may be determined using gel permeation chromatography (GPC), nuclear magnetic resonance spectrometry (NMR), laser light scattering, intrinsic viscosity, vapor pressure osmometry, small angle neutron scattering, laser desorption ionization mass spectrometry, matrix assisted laser desorption ionization mass spectrometry (MALDI MS), or electrospray mass spectrometry or may be obtained from a manufacturer's specifications. Unless otherwise indicated the values of number average molecular weight described herein are determined by gel permeation chromatography (GPC).

In some embodiments, polymers in the core may have any suitable number of repeat units. For instance, the core may comprise one or more polymers having greater than or equal to about 10, greater than or equal to about 20, greater than or equal to about 50, greater than or equal to about 100, greater than or equal to about 200, greater than or equal to about 300, or greater than or equal to about 400. In some instances, the number of repeat units may be less than or equal to about 500, less than or equal to about 400, less than or equal to about 300, less than or equal to about 200, less than or equal to about 100, or less than or equal to about 50. Combinations of the above-referenced ranges are also possible (e.g., greater than or equal to about 10 and less than or equal to about 500). Other values of the number of repeat units in the first component are also possible. The number of repeat units may be determined using gel permeation chromatography (GPC), or nuclear magnetic resonance (NMR), or may be obtained from a manufacturer's specifications.

It should be understood that, in certain embodiments, the particle may be composed of other materials besides synthetic polymers and natural polymers (e.g., polysaccharides, carbohydrates, polypeptides). In certain embodiments, a ceramic such as calcium phosphate ceramic is used. Exemplary calcium phosphate ceramics include tricalcium phosphate, hydroxyapatite, and biphasic calcium phosphate.

In some embodiments, the particles may be biocompatible. For instance, in some embodiments, addition of the particles to cells in vitro results in less than 20% cell death, less than or equal to about 15% cell death, less than or equal to about 12% cell death, less than or equal to about 10% cell death, less than or equal to about 8% cell death, less than or equal to about 5% cell death, less than or equal to about 3% cell death, less than or equal to about 2% cell death, or less than or equal to about 1% cell death and their administration in vivo does not induce inflammation or other such adverse effects.

In general, the particles are biodegradable. As used herein, “biodegradable” particles are those that, when introduced into cells, are broken down by the cellular machinery or by hydrolysis into components that the cells can either reuse or dispose of without significant toxic effects on the cells, i., fewer than about 20% (e.g, fewer than about 15%, fewer than about 10%, fewer than about 5%, fewer than about 3%, fewer than about 2%, fewer than about 1%) of the cells are killed when the components are added to cells in vitro. The components preferably do not cause inflammation or other adverse effects in vivo. In certain embodiments, the chemical reactions relied upon to break down the biodegradable particles are catalyzed. In other embodiments, the chemical reactions relied upon to break down the biodegradable particles are not catalyzed.

The particle may degrade over hours to days to weeks to months, thereby releasing the agent (e.g., pharmaceutically active agent) over an extended period of time. In certain embodiments, the half-life of the particle under physiological conditions is 1-72 hours (e.g., 1-48 hours, 1-24 hours). In certain embodiments, the half-life of the particle under physiological conditions is 1-7 days. In other embodiments, the half-life is from 2-4 weeks. In other embodiments, the half-life is approximately 1 month.

In some embodiments, the particle may be negatively charged. For instance, in some embodiments, the zeta potential of the particle may range from about −1 mV and about −40 mV, range from about −1 mV and about −35 mV, range from about −5 mV and about −40 mV, range from about −10 mV and about −40 mV, range from about −15 mV and about −40 mV, range from about −5 mV and about −35 mV, range from about −5 mV and about −30 mV, range from about −5 mV and about −25 mV, or range from about −10 mV and about −25 mV. In other embodiment, the particles may be neutral, near neutral, or positively charged.

In some embodiments, the particle may optionally contain other components (e.g., chemical compound) in addition to the core and the coating. In some embodiments, the particle may comprise a surface modifying agent attached to the coating via a covalent or non-covalent bond. Examples of surface modifying agents include polymers (e.g., polyethylene glycol) and targeting moieties. In certain embodiments, the surface modifying agents changes the surface characteristics of the particle. Any targeting moiety known in the art of drug delivery may be used in the coating. A variety of targeting moieties that direct pharmaceutical compositions to particular cells are known in the art (see, for example, Cotten et al. Methods Enzym. 217:618, 1993; incorporated herein by reference). Classes of targeting moieties useful in the inventive particles include proteins, peptides, polynucleotides, small organic molecules, metals, metal complexes, carbohydrates, lipids, etc. In certain embodiments, the targeting moiety is a protein or peptide. Antibodies (e.g., humanized monoclonal antibody) or antibody fragment (e.g., Fab fragment) may be used as targeting moieties. In certain embodiments, a protein receptor or a portion of a protein receptor is used as the targeting moiety. In other embodiments, a peptide ligand (e.g. peptide hormone, signaling peptide, peptide ligand, etc.) is used as the targeting moiety. In certain particular embodiments, the targeting moiety is an RGD integrin-binding peptide. In certain embodiments, a peptide aptamer is used. In certain embodiments, the targeting moiety is a glycopeptide or glycoprotein. In certain embodiments, the targeting moiety is an avimer. In certain embodiments, the targeting moiety is a nanobody. In certain embodiments, the targeting moiety is a polynucleotide. In certain particular embodiments, the targeting moiety is DNA-based. In other embodiments, the targeting moiety is RNA-based. In certain embodiments, the targeting moiety is a polynucleotide aptamer. In certain embodiments, the targeting moiety is a carbohydrate. In certain embodiments, the targeting moiety is a carbohydrate ligand. In certain embodiments, the targeting moiety is a carbohydrate found on the surface of a cell. In certain embodiments, the targeting moiety is small molecule. In certain embodiments, the targeting moiety is an organic small molecule. In other embodiments, the targeting moiety is an amino acid. In certain embodiments, the targeting moiety comprises a metal. In certain embodiments, the targeting moiety is an organometallic complex.

In another aspect, methods are provided. In some embodiments, a method of forming a particle comprises coating a core containing one or more pharmaceutically active agents with a biocompatible adhesive polymer or precursor thereof. In certain embodiments, the method comprises coating a core containing an pharmaceutically active agent(s) with one or more monomers that are polymerized on the surface of the core to form a biocompatible adhesive polymer covering at least a portion of the surface of the core. Those of ordinary skill in the art would be knowledgeable of methods to polymerize monomers on the surface of a core. In some instances, the polymerization may be spontaneous or initiated by a suitable initiator (e.g., oxidant, free radical). In certain cases, the monomers may be polymerized at room temperature using non-toxic reagents. For example, a catechol-containing monomer (e.g., dopamine) under spontaneous autoxidation in the presence of oxygen to form a biocompatible adhesive polymer on the surface of the core.

In some embodiments, the monomer may be a catechol-containing molecule. In some such embodiments, the method may comprise providing a polymeric core containing one or more pharmaceutically active agents and polymerizing a catechol-containing molecule on the surface of the polymeric core to form the coated particle. In some embodiments, the catechol-containing molecule comprises the structure:

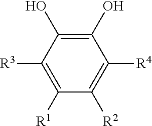

wherein R¹, R², R³ and R⁴ are independently hydrogen, optionally substituted alkyl, optionally substituted alkenyl, amine, acyl, and optionally any two R may be joined to form a ring. or optionally substituted heteroalkyl, and optionally any two R may be joined to form a ring. In certain embodiments, at least one of R¹, R², R³ and R⁴ comprises an amine.

In general, the core may be coated using any methods or techniques known in the art. In certain embodiments, the cores to be coated are prepared separately or are obtained from another source such as a commercial source. In certain embodiments, the cores are prepared and then coated in a seemingly continuous process. The cores may be washed, purified, sized, characterized, etc. before the coating process. The cores may be prepared using any techniques known in the art. Exemplary methods of preparing particles include freeze drying, spray drying, double emulsion, single emulsion, phase inversion, etc. In certain embodiments, the particles are prepared by single emulsion. The cores for coating may also be purchased or provided by a third party.

In some embodiments, the method may further comprise adding an additional component to the particle. For instance, in some embodiments, the method may comprise attaching a surface modifying agent to the surface of the particle. In general, any suitable chemical compound that can be attached to particle. Non-limiting examples of chemical compounds include small molecules, polynucleotides, proteins, peptides, metals, polymers, oligomers, organometallic complexes, lipids, carbohydrates, etc. The chemical compound may modify any property of particle including surface charge, hydrophilicity, hydrophobicity, zeta potential, size, etc. In certain embodiments, the chemical compound is a polymer such as polyethylene glycol (PEG). In certain embodiments, the chemical compound is a targeting moiety used to direct the particles to a particular cell, collection of cells, tissue, or organ system and/or to promote endocytosis or phagocytosis of the particle. Any targeting moiety known in the art of drug delivery may be used.

In another aspect, a particle comprising a solid core coated with a biocompatible adhesive polymer comprising a catecholamine repeating unit is provided. The coating may have one or more biomacromolecules attached to the surface of the coating. In some such embodiments, one or more biomacromolecules may be directly (e.g., covalently, non-covalently) attached to the surface of the coating. That is, in some embodiments, one or more biomacromolecules may be attached to the particle without the use of a linking agent. In some such cases, the coating may further comprise one or more amine groups (e.g., a PEG molecule covalently bonded to one or more amine groups). In some embodiments, the particle may have a higher activity and/or binding affinity than the same biomacromolecule free in solution or otherwise not attached to a material. In certain embodiments, the particles may also have a relatively high stability.

In some embodiments, one or more properties (e.g., all) of the particles comprising one or more biomacromolecules attached to the surface of the coating may be the same as other particles (e.g., particle having a pharmaceutically active agent in a core that is coated with a biocompatible adhesive polymer, particles described above) described herein, except, in some instances, the particle comprising one or more biomacromolecules attached to the surface of the coating may not contain a pharmaceutically active agent in the core. For instance, the particles comprising one or more biomacromolecules attached to the surface of the coating may have a polymer composition of the core, coating composition, structure of the biocompatible adhesive polymer, weight percentage of polymer in the core, biological macromolecule that the particle comprises, particle diameter, core diameter, coefficient of variation in characteristic dimension of a plurality of particles, biocompatibility, and/or zeta potential, amongst other that fall within the ranges and/or description provided above.

In some embodiments, the activity of the biomacromolecule attached to the surface of the coating on the particle may be greater than the activity of the free, or otherwise not attached to a material, biomacromolecule (e.g., a material having at least one cross-sectional dimension greater than the biomacromolecule, a material having a greater size than the biomacromolecule). In some such embodiments, the activity of the biomacromolecule attached to the coating at 25° C. in PBS may be greater than or equal to about 2 times, greater than or equal to about 5 times, greater than or equal to about 10 times, greater than or equal to about 25 times, greater than or equal to about 50 times, greater than or equal to about 75 times, greater than or equal to about 100 times, greater than or equal to about 125 times, greater than or equal to about 150 times, or greater than or equal to about 175 times and less than or equal to about 20 times the activity of the free, or otherwise not attached to a material, biomacromolecule.

In some embodiments, the particle comprising a solid core coated with a biocompatible adhesive polymer comprising a catecholamine repeating unit may be relatively stable during storage at 4° C. and/or 25° C. For instance, in some embodiments, the coefficient of variation in a characteristic dimension (e.g., diameter, cross-sectional dimension) of the particles after storage for at least one week (e.g., two weeks, one month) at 4° C. and/or 25° C. is less than or equal to about 30%, less than or equal to about 28%, less than or equal to about 25%, less than or equal to about 22%, less than or equal to about 20%, less than or equal to about 18%, less than or equal to about 15%, less than or equal to about 12%, or less than or equal to about 10%.

In some embodiments, the percentage of biomacromolecules attached to the surface of the coating that are functional after storage for at least one week (e.g., two weeks, one month) at 4° C. and/or 25° C. is greater than or equal to about 70%, greater than or equal to about 72%, greater than or equal to about 75%, greater than or equal to about 78%, greater than or equal to about 80%, greater than or equal to about 82%, greater than or equal to about 85%, greater than or equal to about 88%, greater than or equal to about 90%, greater than or equal to about 92%, or greater than or equal to about 95%.

In some embodiments, the particle may have a relatively high weight percentage of biomacromolecules attached to the coating. For instance, in some embodiments, the weight percent of total biomacromolecules attached to the surface of the coating is greater than or equal to about 0.5 wt. %, greater than or equal to about 1 wt. %, greater than or equal to about 2 wt. %, greater than or equal to about 3 wt. %, greater than or equal to about 4 wt. %, greater than or equal to about 5 wt. %, greater than or equal to about 6 wt. %, greater than or equal to about 7 wt. %, greater than or equal to about 8 wt. %, or greater than or equal to about 9 wt. %. In some embodiments, the weight percent of total biomacromolecules attached to the surface of the coating is less than or equal to about 10 wt. %, less than or equal to 9 wt. %, less than or equal to about 8 wt. %, less than or equal to about 7 wt. %, less than or equal to about 6 wt. %, less than or equal to about 5 wt. %, less than or equal to about 4 wt. %, less than or equal to about 3 wt. %, or less than or equal to about 2 wt. %. Combination of the above-referenced ranges are also possible (e.g., greater than or equal to about 0.5 wt. % and less than or equal to about 10 wt. %, greater than or equal to about 2 wt. % and less than or equal to about 10 wt. %).

In general, the biomacromolecules may be attached to the surface of the coating via any suitable means. In some embodiments, the biomacromolecule is attached to the surface of the coating without the use of a linking agent. Those of ordinary skill in the art would be knowledge of linking agents. In some such embodiments, the biomacromolecules may be attached to the surface of the coating via one or more covalent bonds (e.g., amide bonds) and/or one or more non-covalent bonds (e.g., hydrogen bonds, electrostatic bonds, van der Waal interactions).

Particles formed via the methods described herein may be particularly useful for administering an agent to a subject in need thereof. In some embodiments, the particles are used to deliver a pharmaceutically active agent. In some instances, the particles are used to deliver to deliver a prophylactic agent. In certain embodiments, the particles are used to deliver diagnostic agents, such as a contrast agent or labelled agent for imaging (e.g., CT, NMR, x-ray, ultrasound). The particles may be administered in any way known in the art of drug delivery, for example, orally, parenterally, intravenously, intramuscularly, subcutaneously, intradermally, transdermally, intrathecally, submucosally, sublingually, rectally, vaginally, etc.

In some embodiments, the particles, formed as described herein, are particularly well-suited for the treatment of proliferative disorders, infectious diseases, autoimmune diseases, inflammatory diseases, and neoplastic disorders (e.g., cancer, benign neoplasm), amongst other. In certain embodiments, the relatively small average diameter of the particles (e.g., less than or equal to about 200 nm) may allow the particles to penetrate deeper into tumors than essentially identical particles that have a larger average diameter.

In some such embodiments, the particles are well suited for treating diseases (e.g., certain cancers) that would benefit from or require multiple pharmaceutically active agents delivered in a single dosage form. For example, a particle for the treatment of proliferative disorders may comprise both a small molecule drug that debulks the tumor and a biological macromolecule (e.g., antibody) that can stimulate the immune system to eradicate any remaining cancer cells, increasing the likelihood of achieving a complete remission. In another example, a particle for the treatment of proliferative disorders may comprise a small molecule anti-cancer agent and a biological macromolecule that degrades the extracellular matrix of the neoplasm to allow the small molecule anti-cancer agent to penetrate deeper into the neoplasm. In other multi-drug applications, particles may contain pharmaceutically active agents that treat different pathologies (e.g., HIV and TB; heart disease and diabetes).

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

In some embodiments, the particles may comprise one or more anti-cancer agents. Anti-cancer agents encompass biotherapeutic anti-cancer agents as well as chemotherapeutic agents.

Exemplary chemotherapeutic agents include, but are not limited to, anti-estrogens (e.g. tamoxifen, raloxifene, and megestrol), LHRH agonists (e.g., goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca2+ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Once the particles have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions of the present invention and for use in accordance with the present invention may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the inventive particles with suitable non irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of an inventive pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the particles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the particles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the particles in a proper medium. Absorption enhancers can also be used to increase the flux of the compound across the skin. The rate can be controlled by either providing a rate controlling membrane or by dispersing the particles in a polymer matrix or gel.

The invention also provides kits for use in preparing or administering the inventive particles or compositions thereof. A kit for forming particles may include cores and a biocompatible adhesive polymer or precursor thereof (e.g., catechol-containing molecule) as well as any solvents, solutions, buffer agents, acids, bases, salts, targeting moiety, etc. needed in the particle formation process. Different kits may be available for different targeting moieties. In certain embodiments, the kit includes materials or reagents for purifying, sizing, and/or characterizing the resulting particles. The kit may also include instructions on how to use the materials in the kit. The one or more agents (e.g., pharmaceutically active agent) to be encapsulated in the particle are typically provided by the user of the kit.

Kits are also provided for using or administering the inventive particles or pharmaceutical compositions thereof. The particles may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the particles or a pharmaceutical composition thereof. Exemplary devices include syringes, spoons, measuring devices, amongst others. The kit may optionally include instructions for administering the inventive particles (e.g., prescribing information).

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

In the compounds and compositions of the invention, the term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C₁-C₁₂ for straight chain, C₃-C₁₂ for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

As used herein, “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl.

The term “heteroalkyl” refers to an alkyl group as described herein in which one or more carbon atoms is replaced by a heteroatom. Suitable heteroatoms include oxygen, sulfur, nitrogen, phosphorus, and the like. Examples of heteroalkyl groups include, but are not limited to, alkoxy, amino, thioester, and the like.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR)SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The terms “amine” and “amino” are art-recognized and refer to both unsubstituted and substituted amines, e.g., a moiety that can be represented by the general formula: N(R′)(R″)(R′″) wherein R′, R″, and R′″ each independently represent a group permitted by the rules of valence. An example of a substituted amine is benzylamine. Another non-limiting example of an amine is cyclohexylamine.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

Any of the above groups may be optionally substituted. As used herein, the term “substituted” is contemplated to include all permissible substituents of organic compounds, “permissible” being in the context of the chemical rules of valence known to those of ordinary skill in the art. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl group” must still comprise the phenyl moiety and cannot be modified by substitution, in this definition, to become, e.g., a pyridine ring. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, aryl, aryloxy, perhaloalkoxy, aralkoxy, heteroaryl, heteroaryloxy, heteroarylalkyl, heteroaralkoxy, azido, amino, halide, alkylthio, oxo, acylalkyl, carboxy esters, -carboxamido, acyloxy, aminoalkyl, alkylaminoaryl, alkylaryl, alkylaminoalkyl, alkoxyaryl, arylamino, aralkylamino, alkylsulfonyl, -carboxamidoalkylaryl, -carboxamidoaryl, hydroxyalkyl, haloalkyl, alkylaminoalkylcarboxy-, aminocarboxamidoalkyl-, cyano, alkoxyalkyl, perhaloalkyl, arylalkyloxyalkyl, and the like.

As used herein, the term “cancer” refers to a malignant neoplasm (Stedman's Medical Dictionary, 25th ed.; Hensyl ed.; Williams & Wilkins: Philadelphia, 1990). The clogP value of a compound, which is the logarithm of its partition coefficient between n-octanol and water log(_{coctanol/cwater}), is a well-established measure of the compound's hydrophilicity. Low hydrophilicities and therefore high log P values cause poor absorption or permeation. It has been shown for a compound to have a reasonable probability of being well absorbed the log P value is less than 5.0.

The following examples are intended to illustrate certain embodiments of the present invention, but do not exemplify the full scope of the invention.

### EXAMPLES

These examples describe the preparation, drug-loading, and biological activity of poly(lactic-co-glycolic acid) (PLGA) based nanoparticles (NP) containing an enzyme or antibody along with small molecule drugs in the same particle. These nanoparticles were coated with polydopamine to improve their stability and release kinetics.

### Example 1

This example describes the formation of PLGA nanoparticles and the attachment of surface modifying agents to the surface of the nanoparticles.

In general, PLGA-based nanometer-sized cores without drugs were formed using a single emulsion technique. Briefly, PLGA (200 mg) or PLGA-PEG was dissolved in 5 mL of dichloromethane (DCM). The polymer solution was added to a 20 mL solution of 5% (w/v) polyvinyl alcohol (PVA) on ice and subsequently sonicated by Misonix (Farmingdale, N.Y.) for 4 min (80% amplitude, 4 sec on, 2 sec off) to generate nano-droplets. For drug and protein (enzyme or antibody) loaded particles the following modifications were made. Polymer cores containing paclitaxel (PTX) and collagenase were formed by dissolving PTX in DCM along with PLGA and collagenase was dissolved in the PVA solution. Polymer cores containing SB-431542 (SB) and E-selectin were formed by dissolving SB in 200 μL ethanol and 5 mL of DCM and adding it to the PLGA solution. E-selectin was dissolved in the PVA solution. The droplets were solidified upon addition to 100 mL DI water under magnetic stirring to form solid polymeric cores. The suspension of the cores was stirred overnight at room temperature to allow complete evaporation of organic solvents before flash freezing and lyophilization.

The solid polymer cores (NP) were coated with polydopamine via the following procedure. The solid polymer cores (400 mg) were resuspended in 29 mL Tris buffer (10 mM, pH 9). Dopamine (30 mg) was dissolved in 1 mL water and added to the NP suspension under vigorous stirring at 4° C. for 3 hours. Polydopamine coated NPs (PD NP) were collected via centrifugation at 10,000 RCF for 1 hour. Afterwards, PD NP pellets were resuspended in 6 mL Tris buffer (10 mM, pH 9).

After coating, the surface of the nanoparticles were modified with polyethylene glycol (PEG). PEGylation was achieved by adding 15 or 30 mg of one of the following aminated-PEG subtypes: i) methoxy-PEG-NH2 (5 kDa), ii) 4arm-PEG-NH2 (5 kDa), iii) NH2-PEG-NH2 (7.5 kDa), iv) 4arm-PEG-NH2 (10 kDa), v) 8arm-PEG-NH2 (20 kDa), vi) Y-PEG-NH2 (40 kDa) to the suspended nanoparticles. The reaction continued under magnetic stirring for 2 hours at 4° C. PEGylated particles (PDP NP) were collected via centrifugation at 10,000 RCF for 1 hours and washed with water via suspension and centrifugation as described previously. Uncoated polymer cores, such as PLGA NPs (P NP), were also PEGylated to form PLGA-PEG NPs (PP NPs), and were collected and washed similar to that of coated particles. In summary, the following polymeric cores and nanoparticles were formed:
1. P NP: PLGA NPs
2. PD NP: Polydopamine coated NPs
3. PP NP: PLGA-PEG NPs
4. PDP NP: PEGylated polydopamine coated NPs
5. The size and surface charge of polymer cores and nanoparticles post-drying were measured by Malvern Zetasizer Nano S series (Worcestershire, UK). The size of the cores are shown in Table 1.

TABLE 1
Polymer core and nanoparticle size, polydispersity, and zeta potential.

| | Size (nm) | pd | Zeta (mV) |
|---|---|---|---|
| PLGA (P) | 163.88 ± 28.51 | 0.11 ± 0.06 | −15.00 ± 9.21 |
| Polydopamine coated | 152.93 ± 12.05 | 0.17 ± 0.08 | −25.00 ± 7.31 |
| (PD) | | | |
| PEGylated | 161.53 ± 1.42 | 0.22 ± 0.02 | −4.50 ± 8.92 |
| polydopamine (PDP) | | | |
| PTX-Collagenase laden | 191.63 ± 7.43 | 0.30 ± 0.11 | −14.30 ± 9.32 |
| PDP | | | |
| SB-Eselectin laden | 182.00 ± 9.04 | 0.12 ± 0.12 | −24.70 ± 3.76 |
| PDP | | | |

### Example 2

This example describes the efficiency of PEGylated nanoparticles modified with different forms of PEG to resist rapid bodily clearance, which was evaluated in vitro. The most suitable PEG for evading body clearance was selected after determining protein adsorption, as a measure of particle opsonization and elimination for particles coated with different PEGs.

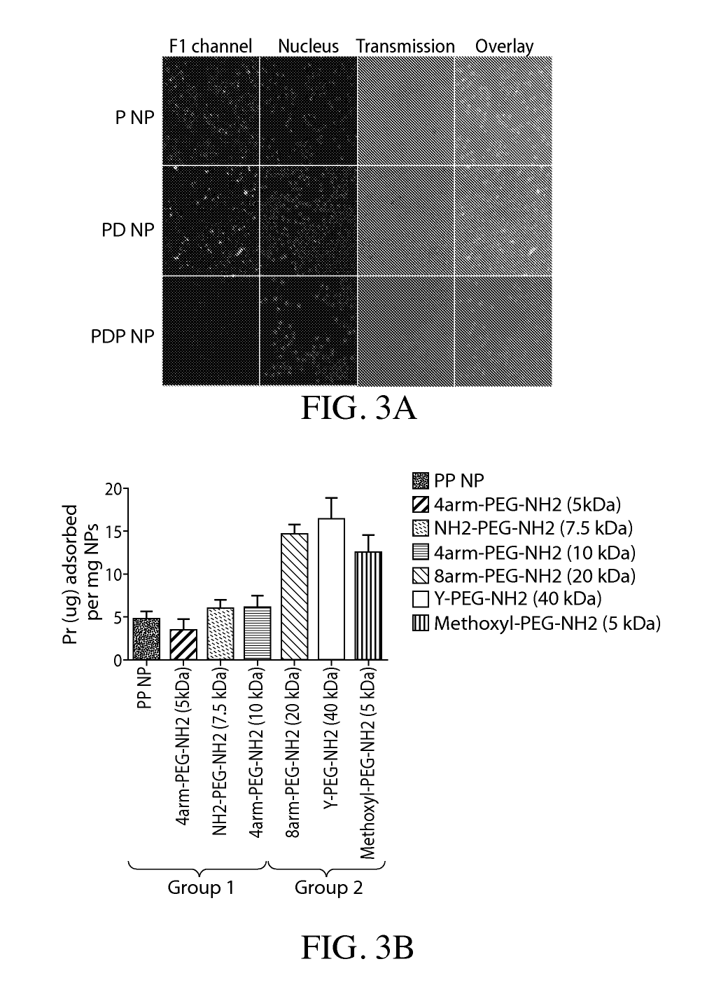

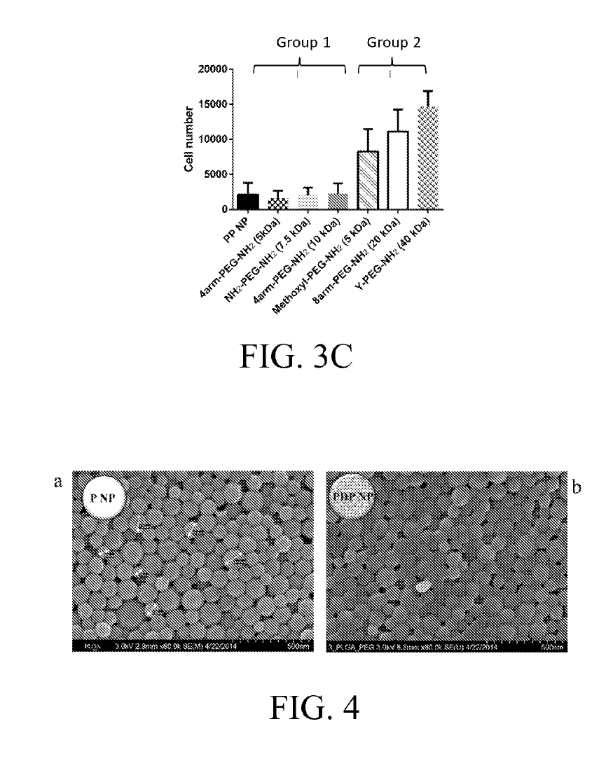

To determine protein adsorption, first, lyophilized fetal bovine serum (FBS) was fluorescently labelled with fluorescein isothiocyanate (FITC). Then, all nanoparticles at a concentration of 10 mg/mL were incubated with DMEM media containing 50% FITC-FBS for 1 hour at 37° C. Nanoparticles were transferred to centrifuge tubes (MWCO 100 kDa) and washed twice with excess PBS. Then, the nanoparticle suspension was transferred to Tube-a-Lyzers with a molecular weight cut off 100 kDa. The nanoparticles were dialyzed in water 3 times with intervals of 4 hours, with the final interval continuing overnight. NPs were then flash frozen and lyophilized. The nanoparticles were resuspended in water at a concentration of 1 mg/mL and fluorescence was measured (485 nm excitation, 535 nm emission) at room temperature using a Spectra Max M5 plate reader (Molecular Devices, Sunnyvale, Calif.). FIG. 3B shows the amount of protein adsorbed to 1 milligram of cores or nanoparticles. Samples can be divided in two groups. Group 1: PP NP, 4arm-PEG-NH₂ (5 kDa), NH₂-PEG-NH2 (7.5 kDa), and 4arm-PEG-NH₂ (10 kDa). Group 2: Methoxy-PEG-NH₂ (5 kDa), 8arm-PEG-NH₂ (20 kDa), and Y-PEG-NH₂ (40 kDa). Group 1 and 2 were significantly different from each other with p values less than 0.001. There was no significant difference within group 1 and 2. Data was expressed as averages with standard deviations of five identically and independently prepared samples. The micrograms of protein adsorbed to 1 mg of each NP was as follows: PP NP=4.8±0.4, Methoxy-PEG-NH2 (5 kDa)=12.6±0.9, 4 arm-PEG-NH2 (5 kDa)=3.6±0.5, NH2-PEG-NH2 (7.5 kDa)=6.0±0.4, 4 arm-PEG-NH2 (10 kDa)=6.2±0.6, 8 arm-PEG-NH2 (20 kDa)=14.6±0.5, and Y-PEG-NH2 (40 kDa)=16.4±1.1. To access, efficiency of PEGylation to resist rapid body clearance was evaluated in vitro, the interaction of the nanoparticles with macrophages in vitro was evaluated. J774A.1 macrophages incubated with plain or PEGylated nanoparticles for 1 hour. PEGylated particles were not taken up by macrophages. FIG. 3A show confocal microscope images of P NP, PD NP, and PDP NP with macrophages. As shown by the images of nanoparticles (F1 channel), nuclei, transmission, and overly images, the PEGylated nanoparticles evaded the macrophages. Among the PEG modifications tested, 4 arm-PEG-NH₂ (5 kDa) most strongly prevented phagocytosis. 2 wt % of PEG was sufficient to result in macrophage evasion. This may be due to a tight PEG loop forming on the NP surface by adherence of the 4 aminated termini per PEG molecule to the NP surface, which could inhibit surface interactions with opsonizing proteins that mark foreign bodies for rapid elimination via phagocytosis. Flow cytometry analysis additionally showed greater uptake of NPs that were coated with PEGs of larger molecular weights (10 kDa and 40 kDa) as shown in FIG. 3C.

Confocal microscopy of particle macrophage interaction was performed as follows. Fluorescently labeled NPs were prepared by replacing 25% of the polymer with fluoresceinamine-conjugated PLGA. Sizes and zeta potential of NPs were measured prior to cell experiments using Malvern Zetasizer Nano ZS series (Worcestershire, UK). J774A. 1 mouse macrophages (ATCC) were grown in DMEM. All media contained 10% fetal bovine serum (FBS) and 100 units/mL penicillin and 100 ug/mL streptomycin. J774A.1 cells were seeded at a density of 50,000 cells/cm² in a 35-mm dish with a glass window (MatTek). After overnight incubation, the medium was replaced with a 0.1 mg/mL NP suspension in serum-free medium and incubated for 1 hour. Cells were then washed with 2 mL of serum-free medium twice to remove free or loosely-bound NPs and before observation using a Leica confocal microscope (Wetzlar, Germany). DRAQ-5 nuclear stain (1-2 μL) was added 2-3 minutes prior to imaging. NPs and cell nuclei were excited using a 488-nm and 633-nm laser respectively. Their emission signals were read from 500 to 600 nm and 650 to 750 nm and expressed in green and blue, respectively (FIG. 1).

### Example 3

This example describes the morphology of the nanoparticles compared to the uncoated polymer cores and the stability of the cores over an extended time period. Morphology was assessed using scanning electron microscopy.

FIG. 4 shows scanning electron microscope images of PLGA-based cores and PEGylated polydopamine coated PLGA-based cores. The PLGA-based cores were formed as described in Example 1 using a single emulsion method and some of the PLGA-based cores were coated with polydopamine and PEGylated as described in Example 1. P NPs and PDP NPS were of sizes below 200 nm and remained stable in suspension for a week at 4° C. P NPs were spherical with smooth surface. Polydopanine deposited on the surface, did not affect the surface smoothness for NPs made of high molecular weight PLGA.

Scanning electron microscopy on cores and nanoparticles were performed as follows. Lyophilized P NPs and PDP NPs were attached to the sample mount using a double-sided conductive carbon adhesive tab and coated with 5 nm platinum with a Cressington HR 208 sputter coater (Cressington Scientific Instruments Ltd, Watford, UK). Samples were imaged with a Hitachi S-4800 field emission scanning electron microscope (Hitachi, Japan) using the high resolution through-the-lens detector (TLD) operating at 3 kV accelerating voltage and -2.9-3 mm working distance (FIG. 2).

### Example 4

This example describes the drug loading of the nanoparticles. For determination of PTX and SB-431542 loading, lyophilized nanoparticles were accurately weighed and then the drugs were extracted using organic solvents and their quantity was measured according to AUC of their respective absorbance peaks compared to standard curve using HPLC. PTX nanoparticles were dissolved in a 50:50 mixture of AcN to water to extract the PTX from the particle. SB-431542 nanoparticles were dissolved in 10:45:45 mixture of ethanol, AcN, and water, respectively, to water to extract the SB-431542 from the particle. Solutions were filtered, and analyzed with high pressure liquid chromatography (HPLC) equipped with Ascentis C18-column (25 cm×4.6 mm, particle size 5 μm). The mobile phase for PTX and SB-431542 were 50:50, and 45:55 AcN:water respectively, with a flow rate of 1 mL/min, respectively. Peaks were detected using a UV detector at 227 nm (PTX), and 325 nm (SB-431542). PTX and SB-431542 content in nanoparticles were calculated as a weight percentage of PTX and/or SB-431542 in nanoparticles.

Table 2 shows the loading of PTX and SB-431542 measured after particle formation and freeze drying. The encapsulation efficiency is determined by the ratio of drug in particles compared to initial added drug prior to particle formation and purification.

TABLE 2
Nanoparticle loading efficiency and encapsulation efficiency.

| | Loading | Encapsulation Efficiency |
|---|---|---|
| | Efficiency (%) | (%) |
| PTX/collagenase-PDP NP | 7.2 ± 1.1 | 85.7 ± 7.3 |
| SB-431542/Eselectin NP | 5.1 ± 0.4 | 66.3 ± 8.9 |

### Example 5

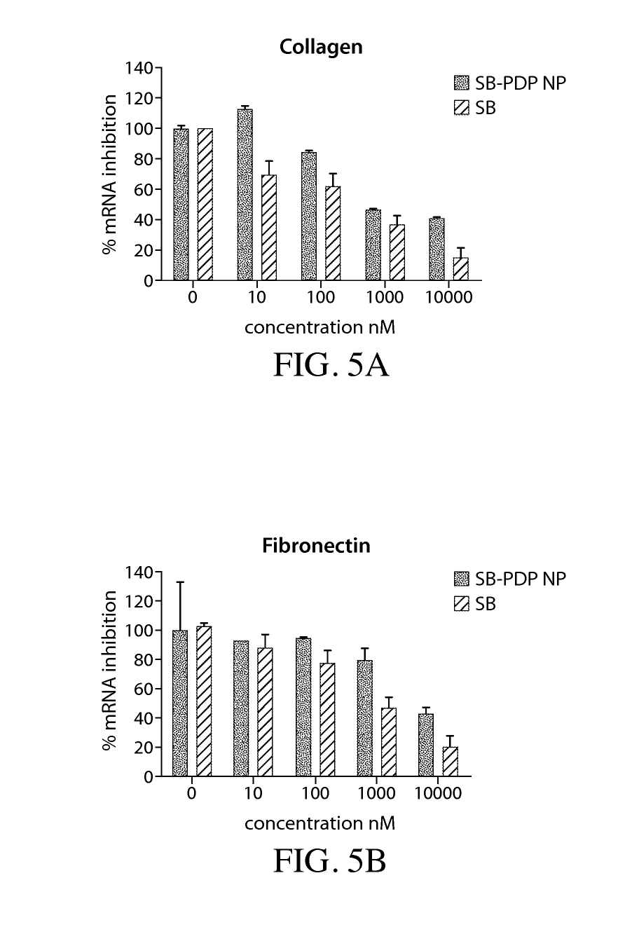

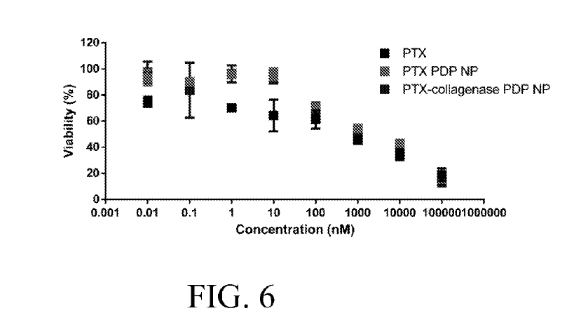

This example describes the activity of drug released from the nanoparticles. As shown in FIG. 5, SB-431542 released from SB-PDP NP was nearly as effective as free drug in at inhibiting TGF-β1-induced extracellular matrix markers. TGF-β1 inhibition of fibroblasts lowered mRNA expression of collagen and fibronectin in a dose-dependent and release-dependent manner. Similarly, as shown in FIG. 6, substantially the same cell viability was observed for SKOV3 and Calu6 exposed to PTX and PTX-laden nanoparticles for 72 hours.

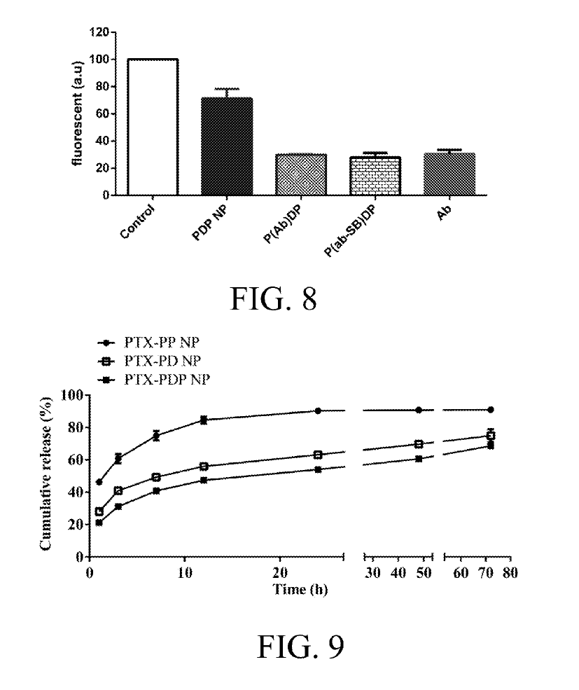

Biological macromolecules released from the particles were also found to have activity similar to or substantially the same as the free drug. PTX/collagenase-PDP NP were found to have collagenase activity of 47.3±6.5. E-selectin antibody was also shown to maintain its activity after being released from the particles. FIG. 8 shows the results of a HLE-60 binding-inhibition assay. The histogram shows the fluorescence of HL-60 bound to HUVEC, which was pre-treated with NPs. Ab on the histograph refers to E-selectin Antibody. The fluorescence obtained with each treatment was normalized to the fluorescence of HL-60 bound to serum-starved medium (SSM)-treated HUVEC. Therefore, NPs or ligands interfering with the HL-60-HUVEC binding resulted in fluorescence lower than 100% (SSM-treated HUVEC, dotted line).

SB-431542 activity was measured as follows. NIH3T3 cells (fibroblast) were seeded into a 96-well assay plate (50,000 cells/well) and grown until confluent at 37° C. with 5% CO2. Cells were then serum-starved for 20-24 hours dosed with SB, m-SB, and SB NPs at concentrations of 10 to 10,000 nM for 4 hours. Same was applied to m-SB-albumin with a dosing of 0.1 to 1 mg/mL. Then, TGF-β was added to a final concentration of 5 ng/ml in each well. Wells with no treatment supplied with TGF-β were considered positive control and no treatment, TGF-β negative were negative controls. Cells were incubated further for another 6 hours. Finally, cells were washed, lysed and the total RNA was extracted using Ambien Cells-to-Ct (Life technologies, USA). cDNA was synthesized using Bio-Rad DNA engine thermal cycler. Relative mRNA expression of collagen and fibronectin was determined using qPCR (Bio-Rad, USA).

PTX activity was measured as follows. Cytotoxicity of PTX and NPs loaded with PTX were evaluated on Calu6 (human lung cancer), SKOV-3 (human ovarian cancer), and BR5FVB1-Akt (murine ovarian cancer) based on the MTT assay. Cells were seeded at a density of 10,000 cells per well in a 96-well and incubated overnight in 200 μL of complete medium. The culture medium was then replaced with 198 μL of fresh medium, two free drug/DMSO solution or NP suspensions in the final concentration ranging from 0.001 to 10,000 nM. After 72 hours of incubation, the medium was replaced with 100 μL of fresh medium containing 13% MTT and incubated for 3.5 hours. Finally, 100 μL of the solubilization/stop solution comprising 20% SDS, 0.02% v/v acetic acid, and 50% v/v DMSO was added to each well, and the absorbance was read at 560 nm by the microplate reader. Cell viability was calculated by dividing the absorbance of treated cells by that of untreated cells after subtracting the absorbance of cell-free medium from each. Here, the untreated cells were those provided with no drug but handled equally otherwise, and the cell-free medium was the medium mixed with MTT solution and stop-solution without cells.

Collagenase activity was determined as follows. A fluorescently labeled gelatin (fl-gelatine 100 μg/mL) was dissolved in 50 mM Tris buffer (pH 7.6, 2 mM sodium azide) in dark. To 100 μL of fl-gelatine solution was added 100 μL standard solutions of collagenase (0.1, 0.05, 0.025, 0.0125, 0.00625, 0.003125 U/mL) or 100 μL of PDPC NP (0.1 mg/mL) in a clear-bottom black 96 well plate. Fluorescence was measured (485 nm excitation, 535 nm emission) after 30 minutes incubation at room temperature using a Spectra Max M5 plate reader (molecular devices, Sunnyvale, Calif.). Nanoparticle collagenase content was determined based on collagenase standard curve.

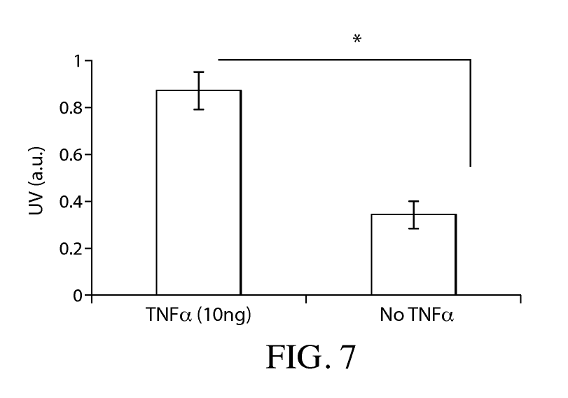

Eselectin activity was determined as follows. First, E-selectin expression was monitored in the presence or absence of TNF (α) as described. Human umbilical vein endothelial cells (passage 3) were plated in 24-well plate coated with collagen type-I. The cells reached confluency within the first 24 h. Then cells were serum starved by exposure to 1 mL Phenol red-free medium 199 (sigma-Aldrich) supplemented with 2 mM L-glutamine and 0.2% (BSA) overnight. Cells were treated with TNF (α) (10 ng/mL) for 4 h. Then incubated with E-selectin Ab for 1 hour at room temperature and consequently washed either once or three times with serum-starved media (SSM) to remove unbound Ab. The secondary Ab (Anti-ab-H.R.peroxidase) was diluted in fresh serum-starved media and added to the cells. After 1 h incubation the secondary Ab was removed and cells were washed either once or three times. TMB (200 uL) was added to each well and kept protected from light for 10 minutes incubation time. HCl acid (1 N, 300 uL) was used as stop solution and the absorbance was read at 450 nm. FIG. 7 shows E-selectin was expressed by 4 hour exposure to 10 ng TNFα.

Then a HL-60 binding-inhibition assay was performed as follows. HUVECs (passage 3) were cultured in a 24-well plate and forced to express E-selectin by exposure to TNFα 10 ng/mL in SSM for 4 hours. The medium was aspirated and cells were treated with one of the following: E-selectin/SB laden nanoparticles, SB-laden nanoparticle, E-selectin-laden nanoparticles, E-selectin Ab (5 μg/mL, positive control), and SSM or PBS as negative controls, for 90 minutes. Meanwhile, HL-60 cells were collected by centrifugation at 1500 rpm for 5 min. Cell pellet was washed with 5 mL PBS to remove the residual medium. For fluorescence microscopy, cells were incubated in Calcein-AM (1 mM, 7 mL) for 45 min and washed with PBS to remove the free dye. The NP treatments were removed from HUVEC cells, which were then incubated with 400,000-500,000 HL-60 cells in 0.5 mL SSM for 60 min at 37° C. Then HUVEC cells were washed 3 times with SSM to remove unbound HL-60 cells and lyzed with 100 μL of cell lysis buffer. The plates were kept in −80° C. fridge overnight, thawed at 37° C. and read with a microplate reader (excitation 485 nm/emission 535 nm). The data was expressed as the average of the fluorescence measured, normalized to the negative controls; either PBS or SSM. The normalized data was expressed in graph and a table as percent fluorescence measured relative to the control. Data were expressed as average±standard deviation of 3-6 independently obtained results per treatment.

### Example 6

This example describes the efficiency of paclitaxel encapsulation in various nanoparticles and their release kinetics.

For release kinetic studies, NPs of a known concentration (1 mg/mL) were resuspended in 1 mL release buffer, consisting of PBS (10 mM phosphate, pH 7.4) containing 0.1% Tween 80, and incubated in a rotating shaker at 37° C. At regular time points (1, 3, 7, 12, 24, 48, and 72 hours), the NP suspension was centrifuged at 10,000 rpm for 10 minutes, and 0.9 mL of the supernatant was collected and replaced with fresh buffer. Loading and encapsulation efficiency was determined as described in Example 4.

PTX was encapsulated in NPs by addition to organic solvent (DCM) during emulsification. All NPs were dried, coated, and/or washed similarly. The yield of NPs produced by coating particles was about 1.5 times higher than NPs produced from linear PLGA-PEG copolymers. Most of the encapsulated PTX was released from PP (copolymer) NPs during washes, resulting in lower loading and encapsulation efficiencies compared to coated NPs. Indeed, dual-coated NPs contained 3.8-fold more PTX than PP NPs. The release kinetics of PTX from NPs was examined in PBS doped with Tween 80 to mimic physiological conditions. Within the first 3 hours, PP NP released 60.8%±2.7 of its PTX payload, whereas coated NPs (PD NP and PDP NP) released 40.8%±2.7 and 31.1%±1.8 PTX, respectively. The coated NPs released the remaining PTX slowly over 72 hours. PTX-PP NP, PTX-PD NP, and PTX-PDP NP samples were significantly different from each other (p<0.05) for each of the given time points.

TABLE 3
Nanoparticle size, polydispersity index, zeta potential, loading
efficiency, and encapsulation efficiency.

| | | | | |
|---|---|---|---|---|
| Size (nm) | pd index | Zeta (mV) | LE (%) | EE (%) |

PLGA MW = 125 kDa

| | | | | | |
|---|---|---|---|---|---|
| PTX-PP NP | 170.9 ± 6.8 | 0.1 ± 0.0 | −11.1 ± 5.1 | 1.9 ± 1.4 | 29.3 ± 9.3 |
| PTX-PD NP | 163.5 ± 1.0 | 0.1 ± 0.0 | −38.3 ± 1.2 | 4.7 ± 3.4 | 55.8 ± 1.2 |
| PTX-PDP NP | 159.0 ± 1.4 | 0.1 ± 0.0 | −10.9 ± 1.1 | 7.2 ± 1.1 | 85.7 ± 3.1 |

PLGA MW = 3 kDa

| | | | | | |
|---|---|---|---|---|---|
| PTX-PP NP | 153.7 ± 4.1 | 0.1 ± 0.0 | −14.1 ± 6.1 | 0.9 ± 2.8 | 10.0 ± 7.1 |
| PTX-PD NP | 144.5 ± 10.0 | 0.1 ± 0.0 | −21.4 ± 2.2 | 1.7 ± 4.4 | 18.9 ± 5.2 |
| PTX-PDP NP | 159.0 ± 1.4 | 0.1 ± 0.0 | −10.9 ± 1.1 | 5.1 ± 6.4 | 57.0 ± 12.1 |

PTX-PDP NPs contained 3.8-fold more PTX than NPs formulated from linear PLGA-PEG copolymer (PTX-PP NP). Since the number of purification steps was the same for both PDP and PP NPs, enhanced drug loading could be attributed to the dual surface coating. The data suggested that the polydopamine layer, along with the 4 arm-PEG-NH₂ layer, on the surface of the particles formed a surface barrier that reduces drug loss during purification steps and extends drug retention in vitro and in vivo. The high loading efficiency achieved with this system reduced the quantity of particle dose required, as the amount of excipient in the formulation was reduced.

Importantly, it was observed that the dual-layer coating with polydopamine and PEG not only improved loading efficiency but also extends the release profile of PTX compared to conventional PP NPs. The surface coating of PTX-PDP NPs reduces the cumulative release of PTX by 1.9-fold in the first 7 hours (41% PTX released in PTX-PDP NP) and sustained the release for 3 days. In contrast, 75% of PTX is released from traditional PP NPs in the first 7 hours, and the remainder was released within one day.

### Example 7

This example describes the time-dependent cytotoxic effects of free PTX, PTX-PP NPs, PTX-PD NPs, and PTX-PDP NPs on BR5FVB1-Akt (mouse ovarian cancer), SKOV3 (human ovarian cancer), and Calu6 (human lung cancer) cells. When cells were exposed to free PTX or drug-laden NPs for 72 hours, the dose response was similar. The IC₅₀ of PTX in SKOV3 and Calu6 cells was the same (˜10 nM), however BR5FVB1-Akt cells were 10-fold more resistant to PTX, with an IC₅₀ of ˜100 nM.

Cell viability was determined as described in Example 5.

When cells were exposed to free PTX or NPs for a short period of time (3 hours), a significant difference in cytotoxicity pattern emerged. Unlike for free PTX and PD NPs, toxicity was substantially reduced for PDP NPs. A decrease in toxicity was similarly observed following acute exposure to PP NPs, but to a much lesser extent.

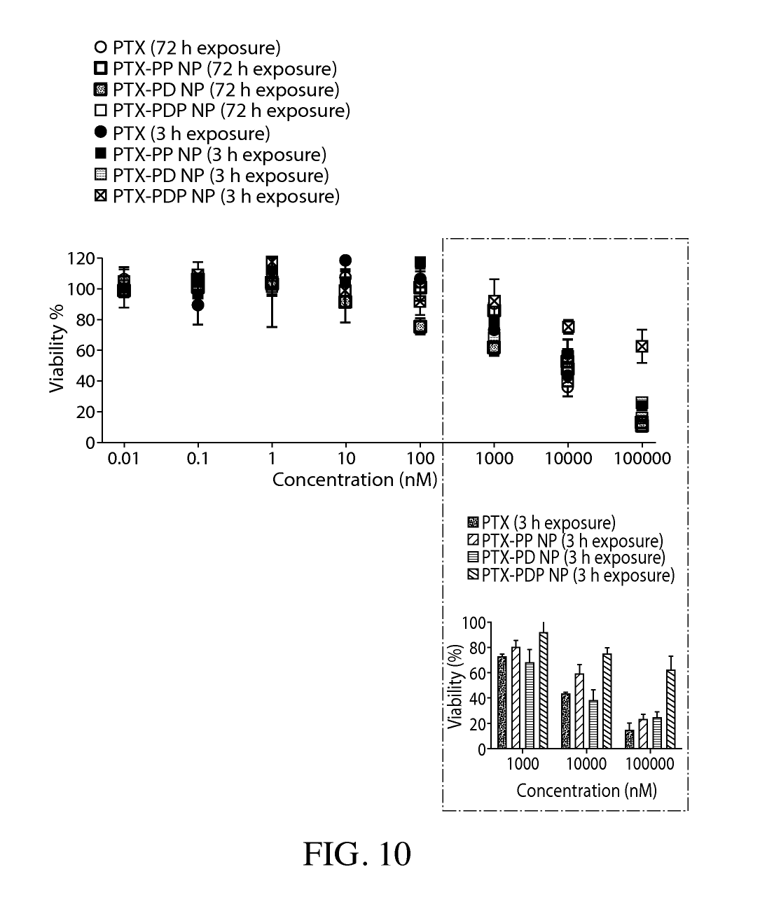

With respect to BR5FVB1-Akt cells, PTX present in the media was quickly taken up by cells and killed cells irrespective of additional exposure time as shown in FIG. 10. PTX-PP NPs released the drug quickly and thus exhibited similar behavior to free drug. PTX-PD NPs, which were coated with a cellular adhesive material, maintained contact with cells after media substitution (after 3 hours) and continued the drug release. In accordance with their in vitro release profiles, PTX-PDP NPs exhibit limited cytotoxicity after the short exposure, as only 30% of their PTX payload was released in the first 3 hours.

Moreover, PTX was released from NPs results in cellular cytotoxicity in a dose-dependent manner. PTX release was time dependent, so BR5FVB1-Akt cells exposed to free PTX or PTX-laden NPs for 72 hours (when total PTX release is achieved) exhibit the same dose-response curve. In contrast, cells exposed to PTX-PDP NP for a short amount of time (3 hours) had lower toxicity compared to free PTX, PTX-PD NPs or PTX-PP NPs as shown in FIG. 10. This result correlated with the observed release profile, with the exception of PTX-PD NPs. The short-exposure (3 hours) cytotoxicity of PTX-PD NP could be attributed to polydopamine-related cellular adhesion that allowed the NPs to remain with the cells and continue PTX release even following media exchange.

### Example 8

This example describes the release of PTX and nanoparticles containing PTX in mice via measurement of the PTX content in murine blood and peritoneal cavity.

Six week-old female FVB mice were obtained from Charles River Laboratory (Boston, USA). Mice were housed in a pathogen-free facility in accordance with the standards of the Dana-Farber Cancer Institute. All animal experiments were approved by the Institute's IACUC. Mice were kept in groups of 4-5 per cage in a 12 hour light/12 hour dark cycle and housed at 25° C. with 50% relative humidity. Mice (n=4 per group) were given IP doses of 5 mg/kg free PTX or PTX-PDP NP as a rapid bolus. Mice were anaesthetized, and blood samples were collected via retro-orbital bleeding 3 hours post dosing. Blood samples were kept on ice until centrifugation. Samples were centrifuged at 1,400 RCF for 10 min, and plasma was collected. After blood sample collection, mice were sacrificed, and their peritoneal cavities were washed thrice post-mortem with PBS. Plasma and peritoneal washes were flash frozen with liquid nitrogen and lyophilized. The lyophilized powders were rehydrated in 300 μL water and extracted with tert-butyl methyl ether (tBME). The organic solvent was dried under nitrogen. The samples were resuspended in a 50:50 mixture of ACN and water and analyzed by HPLC as described Example 4. p<0.001, for PTX measurements in peritoneum; and **: p<0.02 for blood samples (Tukey's test).

The in vitro release profile and in vivo PTX distribution was validated by comparing drug retention in the peritoneal cavity following IP administration of PTX-PDP NPs or free PTX as shown in FIG. 11. To quantify PTX concentration in murine blood and peritoneal cavity, samples were collected via retro-orbital bleeding (pre-mortem) and peritoneal lavage (post-mortem). Mice that received free PTX had three times as much PTX in their systemic circulation (9.5±1.9 μg/mL) as mice treated with PTX-PDP NPs (3.0±1.8 μg/mL). In contrast, an ˜8-fold larger dose of PTX (32.8±9.5 μg/mL) was detected in the peritoneal cavities of mice when PTX-PDP NPs were administered compared to when free PTX was administered (4.5±1.3 μg/mL). Together, these data demonstrate that PTX-PDP NP can be retain drug locally in the peritoneal cavity.

### Example 9

This example describes the IP tumor development and subsequent survival study in an immunocompetent murine model of ovarian cancer.

The BR5FVB1-Akt mouse ovarian cancer cell line was cultured in DMEM supplemented with 10% FBS and 1% penicillin/streptomycin at 37° C. with 5% CO₂. Subconfluent BR5FVB1-Akt cells were trypsinized, washed, and suspended in opti-MEM as a single-cell suspension. Six week-old female FVB mice were injected IP with a total volume of 400 μl opti-MEM containing 3.75×10⁶ BR5FVB1-Akt cells.

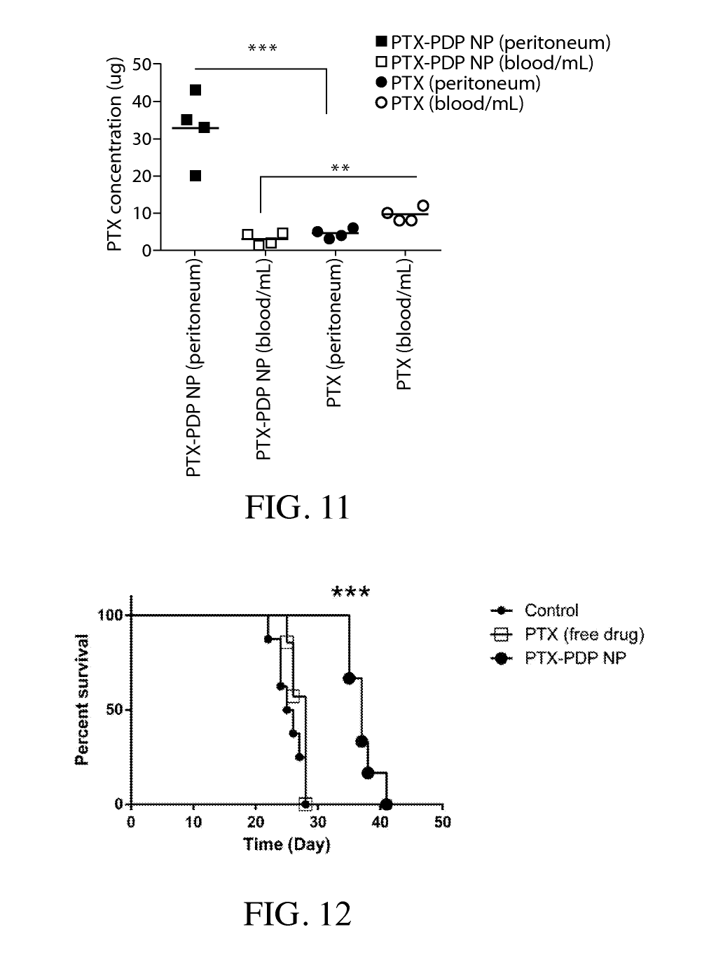

Four days after IP inoculation of cancer cells, the mice (n=8) were treated with PTX (5 mg/kg dissolved in 50 μL DMSO and 450 μL PBS), PTX-PDP NP (5 mg/kg in PBS) or controls (DMSO/PBS mix or PDP NP in PBS) twice weekly for total of five doses. The mice, which received no further treatment, were subsequently monitored for health. Mice were sacrificed by CO₂ asphyxiation upon ascites development. Their peritoneal cavities were opened postmortem to observe tumor location and extent of metastasis in the peritoneal region. All FVB mice inoculated with BR5FVB1-Akt cells developed tumors. Tumors developed gradually and manifested by formation of ascites in the peritoneal cavity. Tumors were detectable post-mortem as nodules of various sizes and number on the peritoneal wall or adhered to the intestine. Control groups (drug-free NPs and DMSO:PBS) and mice that received free PTX (5 mg/kg) developed tumors at similar time points, during the fourth week after tumor cell injection. PTX-PDP NP significantly improved survival, as tumor onset in mice treated with these particles was delayed by one to two weeks as shown in FIG. 12. Aside from the therapeutic benefits, no weight loss or other visible side effects were observed in mice treated with PTX-PDP NP.

Statistical analysis was performed with GraphPad Prism 5 (La Jolla, Calif., USA). Statistical significance among groups was determined by one-way ANOVA, and pairs were compared by Tukey's multi-comparison test. Survival groups were compared using a Mantel-Cox test. Samples were considered to be significantly different if p<0.05.

### Example 10

This example describes the formation of the particles in Examples 10-14.

PLGA nanoparticles were formed as described in Example 1. About 400 mg of nanoparticles were resuspended in 29 mL Tris buffer (10 mM, pH 9). About 30 mg of dopamine was dissolved in 1 mL water and added to the nanoparticle suspension under vigorous stirring at 4° C. for 3 hours. Polydopamine-coated NPs (PD NP) were collected via centrifugation at 10,000 RCF for 1 hour. Afterwards, PD NP pellets were resuspended in 6 mL Tris buffer (10 mM, pH 9), and nanoparticle size was measured using Malvern Zetasizer Nano S series, Worcestershire, UK. Surface coating with proteins was achieved by simultaneous addition of one of the following proteins i) lysozyme, ii) DNase, iii) collagenase I, or iv) E-selectin antibody and 4 arm-PEG-NH₂ (5 kDa). For all proteins, a ratio of protein weight to nanoparticle weight of 1:100 was selected. The reaction continued under magnetic stirring for 2 hours at 4° C. Nanoparticles were collected via centrifugation at 10,000 RCF for 1 hour and washed with water via suspension and centrifugation as described above. Nanoparticle size and surface charge were measured using a Malvern Zetasizer Nano S series (Worcestershire, UK).

In summary, the following nanoparticles were formulated:

P NP: PLGA NPs

PD NP: Polydopamine-coated NPs

PDP NP: Polydopamine- and PEG-coated NPs

PDP-lysozyme: Polydopamine, PEG, and lysozyme-coated NPs

PDP-DNase: Polydopamine, PEG, and DNAse-coated NPs

PDP-Collagenase: Polydopamine, PEG, and Collagenase-coated NPs

PDP-Ab: Polydopamine, PEG, and E-selectin antibody-coated NPs

Dox-PDP-Collagenase: Doxorubicin-laden, Polydopamine, PEG, and Collagenase-coated NPs

### Example 11

This example describes the stability of the polydopamine coated nanoparticles containing proteins on the surface of the particle. Approximately 80% of all nanoparticle types did not aggregate after one month of storage at 4° C.

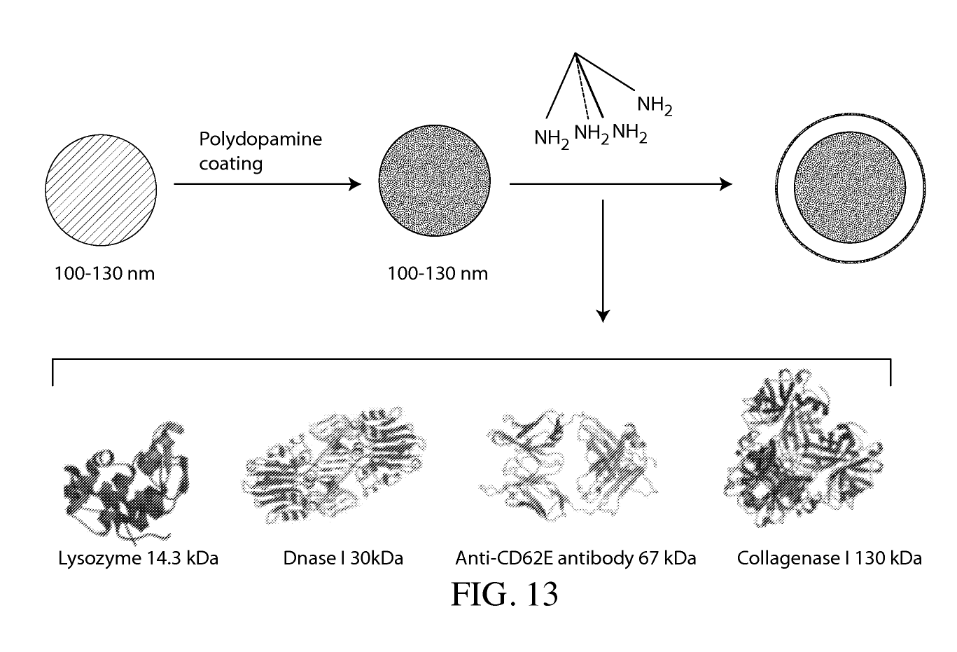

Nanoparticles or proteins are often formulated as suspensions. While, suspensions are expected to remain un-aggregated or responsive to re-suspension during shelf-life, many nanoparticles and proteins in suspension tend to aggregate over time. To test the stability of surface-coated particles, the extent of nanoparticle aggregation at 4° C. under regular storage for proteins was measured for one month 4° C. The extent of aggregation was determined by measuring particle size over time using a Malvern Zetasizer Nano S series (Worcestershire, UK) and well as determining the percentage of particles still in suspension. Storage at 25° C. was also determined. FIG. 13 shows a schematic of particle formation.

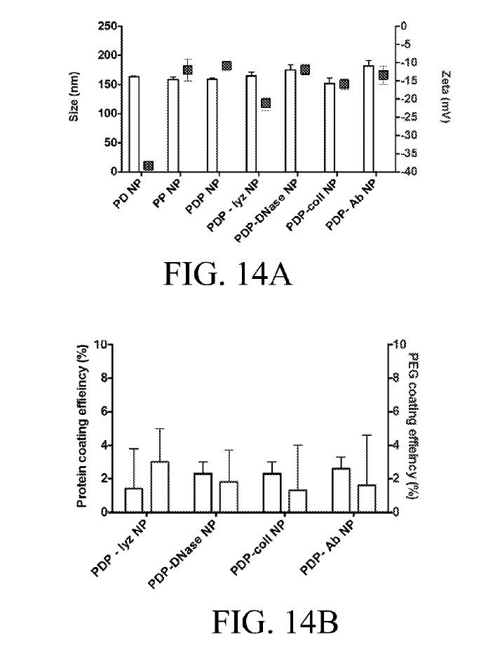

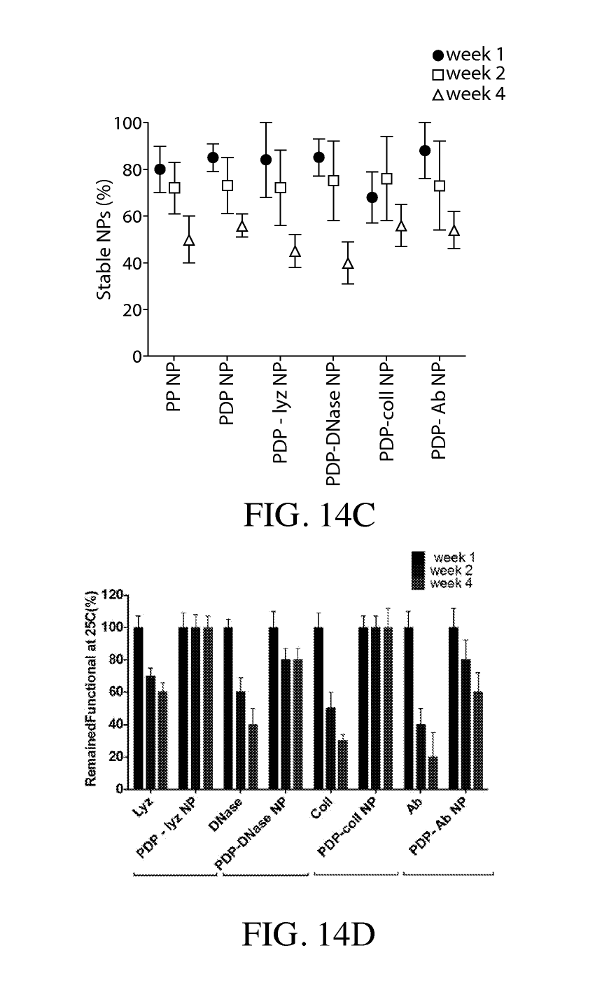

Approximately 80% of all nanoparticle types did not aggregate after one month of storage at 4° C. Particles coated with polydopamine and an outer layer of proteins had substantially the same stability as particles lacking proteins on the surface and even substantially the same as particles having a higher PEG concentration (e.g., PLGA-PEG in FIG. 14C) of higher PEG concentration.

Next, the stability of nanoparticles and their soluble protein counterparts at room temperature was tested. Within a week all particles were aggregated and colloids were observed in the protein solutions. However, unlike the soluble protein that lost functionality, aggregated particles retain their functionality.

FIG. 14A shows nanoparticle size before and after coating, demonstrating that nanoparticle size did not alter during and after surface coating with proteins. Surface charge was a function of PEGylation and was not affected by proteins' initial charge. FIG. 14B shows that the weight percentage of protein and PEG coating in the particles were almost equivalent. FIG. 14C shows the percent of stable nanoparticles for various nanoparticles over time. The nanoparticles remained relatively stable in size for two weeks at between about 2-8° C. Though aggregation gradually occurred, the particles retained their functionality irrespective of the size change for 3 months at 4° C. FIG. 14D shows the percentage of particles that remained function after one week at 25° C. In general, the proteins still retained their functionality compared to proteins in solution under the same condition. The weight percentage of protein and PEG on the surface of the coating were almost equivalent; both were about 2%.

### Example 12

This example describes the in vitro activity of proteins on the surface of polydopamine coated particles. Proteins coated on the surface of the particles had enhanced biological function in vitro.

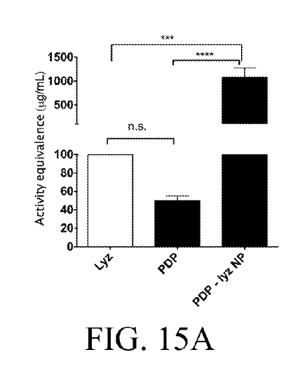

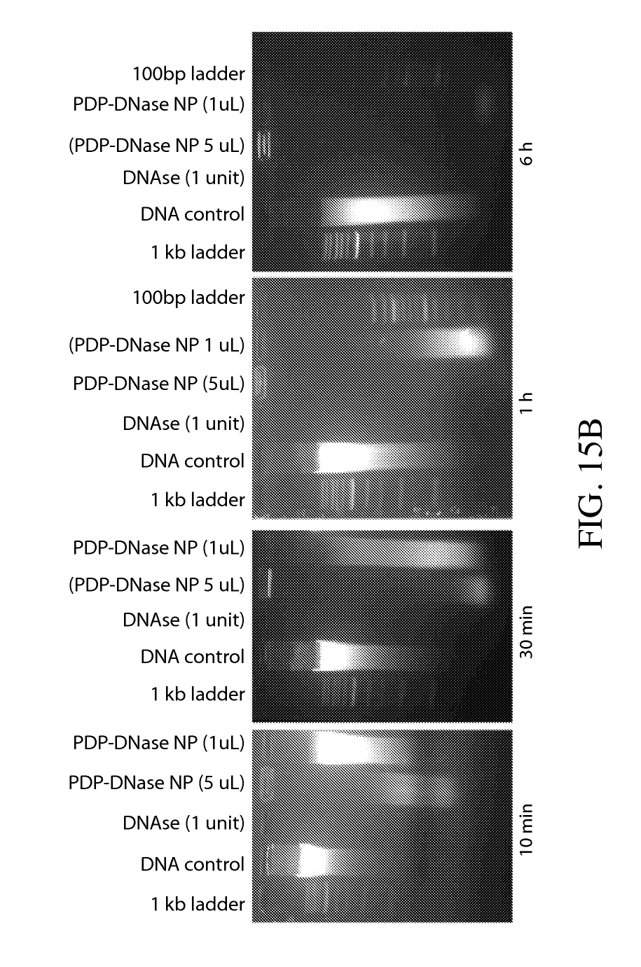

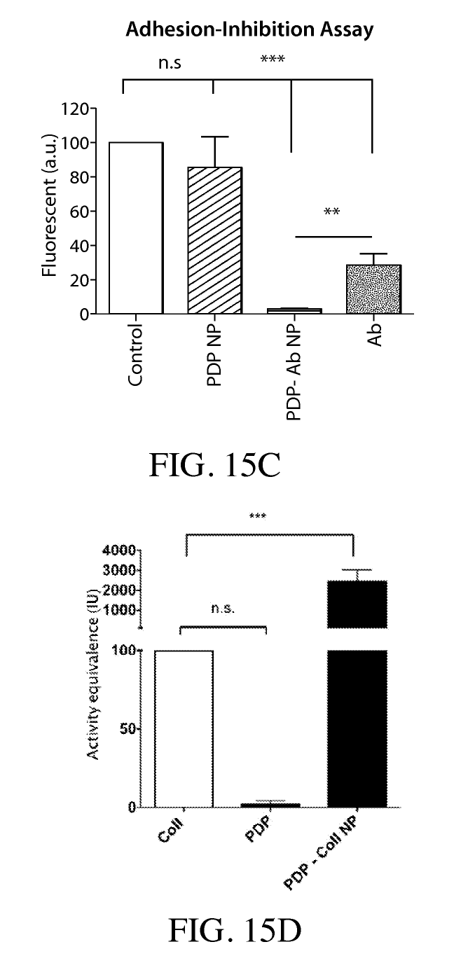

In vitro functional assays tailored to individual proteins were conducted. Lysozyme's ability to degrade M. lysodeikticus can be correlated to its functionality. To determine the functionality of lysozyme on the particles and in solution, a kinetic standard curve based on reduced turbidity of known bacterial suspension was established. Then, the extent of reduced turbidity upon exposure to lysozyme-coated nanoparticles and lysozyme in solution was measured over time. Compared to lysozyme in solution, surface bound lysozyme was about 10 times more potent as illustrated in FIG. 15A.

Next, the ability of surface bound DNAse to degrade highly polymerized DNA was investigated. Interestingly, PDP-DNase NPs digested DNA with the same efficiency as DNAse in solution, but in a sustained manner. The impact of sustained DNA degradation on cellular components was also investigated. The addition of DNAse in solution to media of highly aggressive triple negative breast cancer models (4T1) did not cause any toxicity to the cells.

Among proteins, antibodies function in a highly specific and coordinated manner. Therefore, a process, which alters the specific paratope would render the antibody ineffective. Thus, if nanoparticle surface-bound antibodies may alter the respective paratope was investigated. Anti-CD62L antibody, which bind to CD62L receptor in the same manner as Sialyl Lewis x (sLex) receptor's ligand was chosen. Human Umbilical Vein Endothelial Cells (HUVEC) expression of CD62L under stress was used to test the functionality of the nanoparticles. The stress response was mimicked by exposing HUVEC with TNF α. Then, fluorescently labeled anti-CD62L antibody or PDP-ab NPs was incubated with HUVEC for 2 hours. After washing, the level of fluorescence was attributed to adhesion.

In addition to adhesion, the ability of PDP-ab NP to compete with CD62L was also studied in a complementary study. Stimulated HUVECs were first incubated with anti-CD62L or PDP-ab NPs for two hours, followed by fluorescently-labeled HL-60, which are leukocytes that naturally express sLex. The ability of antibody to inhibit adhesion of the competing species, here HL-60, showed the receptor's binding potency. Interestingly, surface binding of anti-CD62L antibody did not reduce the antibody function but also enhanced binding ability by 10 fold. This result may be attributed to multivalency. Finally, to evaluate the versatility of this methodology, a larger protein collagenase I (130 kDa) was tested. The nanoparticle having collagenase on the surface (i.e., PDP-Col I NP had an increased potency of collagen I degradation by 24.5±5.8 fold compared to collagenase in solution.

FIG. 15A shows a graph of activity equivalence for lysozyme coated nanoparticles, polydopamine nanoparticles, and lysozyme in solution. For the lysis of Micrococcus lysodeikticus, lysozyme coated nanoparticles had a higher efficiency compared to lysozyme solution. FIG. 15B shows gel electrophoresis images for DNase nanoparticles at a dosage of equivalent to 1 unit DNase and 5 unit DNase and DNase in solution. The DNase nanoparticles degraded highly polymerized DNA with the same efficiency as DNase in solution, but over an extended time. FIG. 15C shows the results of the adhesion-inhibition assay for anti-CD62E antibody coated nanoparticles, polydopamine nanoparticles, and anti-CD62E antibody in solution. Anti-CD62E coated nanoparticles blocked adherence of leukocytes (HL-60) to HUVEC expressing CD62E with higher potency than anti-CD62E antibody in solution. FIG. 15D shows a graph of activity equivalence for collagenase I coated nanoparticles, polydopamine nanoparticles, and collagenase I in solution. Collagenase I coated nanoparticle degraded collagen solution with extensively higher efficiency than that of collagenase I solution.

### Example 13

This example describes an in vivo functional assay for the collagenase coated nanoparticles in Examples 12. The collagenase coated nanoparticles reduced tumor size in vivo.

Part of tumor scaffold in many cancers (e.g. breast and ovarian) is made of collagen deposited by fibroblasts. This scaffold promotes attachment, survival and proliferation of cancer cells and stromal cells. Degradation of tumor scaffold should allow for enhanced penetration of anti-cancer drugs to the tumor and thus reduced tumor burden. Therefore, PDP ColI NPs (intratumorally, 20 IU) was injected to BalbC mice bearing 4T1 breast tumors having a size of about 1500 mm².

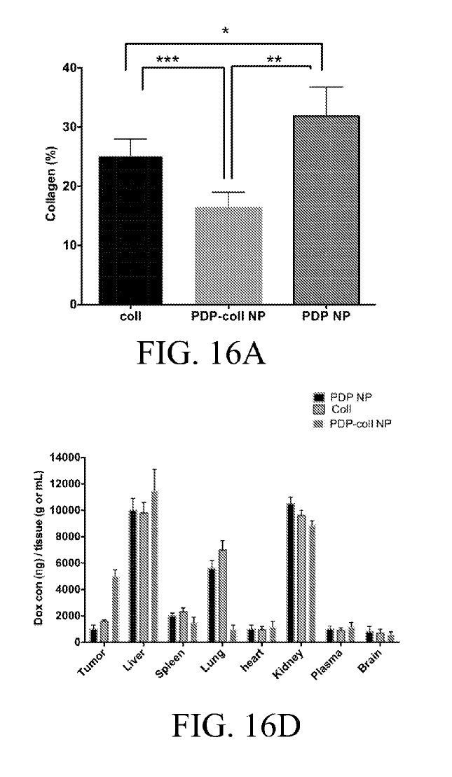

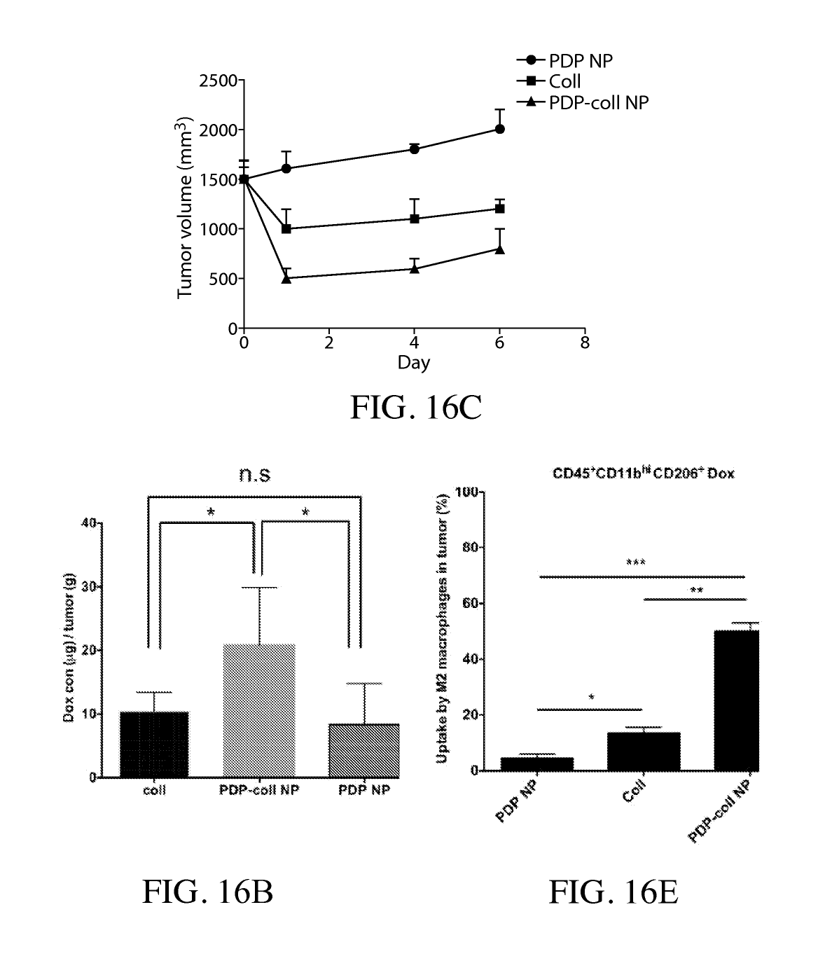

PDP ColI NPs reduced the tumor's collagen content to 50% of its initial concentration and consequently reduced the tumor burden to ⅓ overnight as shown in FIG. 16. The reduction in collagen concentration and tumor burden was also observed for collagenase in solution, but the collagenase in solution was only half as effective as the collagenase coated nanoparticles.

Unfortunately, due to the aggressive nature of these triple negative breast cancers, the tumors regressed rapidly. Thus, there is a need to combine collagenase therapy with an anti-cancer agent such as doxorubicin (Dox) to enhance therapeutic potential. In this context and to understand whether lowering the tumor matrix would enhance delivery of Dox, Dox was injected intraperitoneally, after the tumor burden was lowered using PDP ColI NPs. Biodistribution study, shown in FIG. 16E, doxorubicin accumulation in the tumor was higher when PDP ColI NPs were used as opposed to collagenase in solution. The nanoparticles had a 3.1±0.5 fold increase in doxorubicin accumulation compared to soluble collagenase I of the same potency. Interestingly, the majority of Dox was detected in immunosuppressive macrophages CD45⁺CD11b⁺F4/80⁺GR1⁻MHCII^{lo}CD206^{hi} of alternative phenotype (M2).

FIG. 16A shows the percent collagen in mice having triple negative breast cancer after intratumoral injection of collagenase I coated nanoparticles, collagenase in solution, and polydopamine coated nanoparticles. FIG. 16B shows a graph of tumor volume versus number of days for collagenase I coated nanoparticles, collagenase in solution, and polydopamine coated nanoparticles. FIG. 16C shows the doxorubicin concentration in triple negative breast cancer tumors in mice for collagenase I coated nanoparticles, collagenase in solution, and polydopamine coated nanoparticles. FIG. 16D shows the doxorubicin concentration in various tissues of mice having triple negative breast cancer for collagenase I coated nanoparticles, collagenase in solution, and polydopamine coated nanoparticles. Disrupting the tumor matrix enhanced accumulation of Dox in the tumor and decreased lung accumulation, while Dox distribution in other organs remained unchanged. FIG. 16E shows the uptake of doxorubicin in M2 macrophages in triple negative breast cancer tumor in mice for collagenase I coated nanoparticles, collagenase in solution, and polydopamine coated nanoparticles. Higher quantities of doxorubicin were present in M2 immunosuppressive macrophages in the tumor-microenvironment.

### Example 14

This example describes the release characteristics of coated nanoparticles comprising doxorubicin in the nanoparticle core. Coated nanoparticles had a lower release of doxorubicin than uncoated nanoparticles, resulting in continuous exposure of cancer cells to anti-cancer drugs and thus lower resistance.

Nanoparticles with doxorubicin in the core were formed as described in Example 1, except doxorubicin was added to the organic solvent. The particles were coated as described in Example 10. To test the cell viability for cells exposed to doxorubicin particles, 500 ng/mL of Dox or Dox-laden nanoparticles were added to matrigel on top chamber of a 24 well plates seeded with 4T1 cells. Plates were incubated at 37° C., while gently shaken. The viability was measured at 3 hours, 6 hours, and 24 hours. Cell viability was determined as described in Example 5. For the mammosphere assay, 4T1 cells were seeded at 1000 cells/150 uL/well in 96 well ultra-low adherence plate. The remaining 4T1 cells after treatment were harvested and filtered and the number of mammospheres counted using microscopy.

In addition to using the surface of nanoparticles as carriers for delivery of proteins, the core was used to load Dox. A reduced burst release was observed for coated nanoparticles, indicating that the surface coating functioned as a barrier toward release of the drug. Ultimately, this extended release increased the exposure time for the cells to a non-degraded form of Dox compared to doxorubicin in solution and uncoated nanoparticles and lowered the formation of mamospheres, which is an indication of resistant cells due to prolonged chemotherapy. Thus, surface modulation of nanoparticles not only provided a mean to enhance protein function and/or efficiency, but also formed a surface barrier to protect drug from rapid release or degradation.

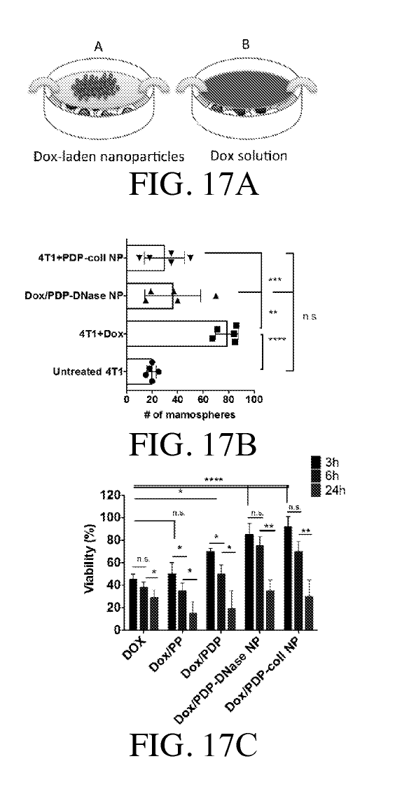

FIG. 17A shows a schematic of the experimental method for nanoparticles comprising doxorubicin and doxorubicin in solution. FIG. 17B shows cell viability for Dox in solution, uncoated nanoparticles comprising Dox, polydopamine nanoparticles comprising Dox, DNase nanoparticles comprising Dox, and collagenase I nanoparticles comprising Dox at various times. Dox in solution and Dox loaded in nanoparticles made of linear PLGA-PEG polymer were released shortly and reduced viability of cells. Coated nanoparticles released Dox slower. FIG. 17C shows the number of mammospheres for untreated 4T1 cells, Dox in solution, DNase nanoparticles comprising Dox, and collagenase I nanoparticles comprising Dox. The reduced number of mammospheres in sustained-release formulations is indicative of a lower likelihood of developing drug resistance following chemotherapy.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A nanoparticle comprising:
a polymeric core containing an pharmaceutically active agent; and
a coating covering the surface of the polymeric core, wherein the coating comprises a biocompatible adhesive polymer.

2. A nanoparticle as in claim 1, wherein the pharmaceutically active agent is a biological macromolecule.

3. A nanoparticle as in claim 1 further comprising a second pharmaceutically active agent.

4. A nanoparticle as in claim 1, wherein the biocompatible adhesive polymer comprises a catechol-containing repeating unit

5. A nanoparticle as in claim 1, wherein the catechol-containing repeating unit is a catecholamine repeating unit.

6-13. (canceled)

14. A nanoparticle as in claim 1, wherein the biocompatible adhesive polymer is polydopamine.

15. A nanoparticle as in claim 2, wherein the biological macromolecule is an enzyme.

16. A nanoparticle as in claim 15, wherein the enzyme is an extracellular matrix degrading enzyme.

17-21. (canceled)

22. A nanoparticle as in claim 4, wherein the catechol-containing repeat unit comprises the structure: 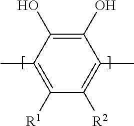
wherein:
R¹ and R² are independently hydrogen, optionally substituted alkyl, optionally substituted alkenyl, amine, acyl, or optionally substituted heteroalkyl; and
optionally R¹ and R² may be joined to form a ring.

23. (canceled)

24. A nanoparticle as in claim 5, wherein the catecholamine repeat unit comprises the structure:

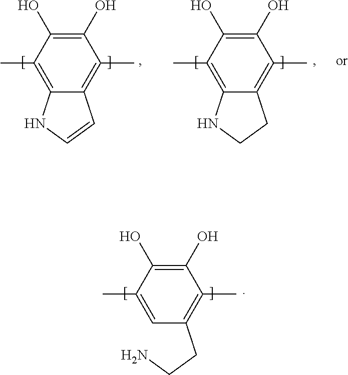

25-31. (canceled)

32. A nanoparticle as in claim 1, wherein the polymeric core comprises a synthetic polymer.

33-38. (canceled)

39. A nanoparticle as in claim 1 comprising a surface modifying agent attached to the coating.

40-42. (canceled)

43. A composition comprising:
a plurality of nanoparticles of claim 1; and
one or more excipients.

44. A pharmaceutical composition comprising:
a therapeutically effective amount of the nanoparticles of claim 1; and
one or more pharmaceutically acceptable excipients.

45. A method of treating proliferative disorder in a subject comprising:
administering a pharmaceutical composition as in claim 43 to the subject.

46-47. (canceled)

48. A method of administering a nanoparticle comprising:
administering the nanoparticle of claim 1 to a subject.

49-50. (canceled)

51. An in vitro method of administering a nanoparticle to a cell, comprising:
administering the nanoparticle of claim 1 to a cell.

52-56. (canceled)

57. A method of forming a coated nanoparticle comprising:
providing a polymeric core containing an pharmaceutically active agent; and
polymerizing a catechol-containing molecule on the surface of the polymeric core to form a nanoparticle as in claim 1.

58-60. (canceled)

61. A nanoparticle comprising:
a solid core;
a coating covering the surface of the polymeric core, wherein the coating comprises a biocompatible adhesive polymer comprises a catechol-containing repeating unit; and
a biomacromolecule directly attached to the surface of the coating.

62-108. (canceled)
